(19) United States
(12) Patent Application Publication
(10) Pub. No.: US 2021/0017206 A1
(43) Pub. Date: Jan. 21, 2021

(54) PYRIDINONE AND PYRIMIDINONE PHOSPHATES AND BORONATES USEFUL AS ANTIBACTERIAL AGENTS

(71) Applicant: Pfizer Inc., New York, NY (US)

(72) Inventors: Matthew Frank Brown, Stonington, CT (US); Ye Che, Niantic, CT (US); Anthony Marfat, Mystic, CT (US); Michael Joseph Melnick, Portage, MI (US); Justin Ian Montgomery, Ledyard, CT (US); Timothy Allan Johnson, Vicksburg, MI (US); Richard Andrew Ewin, Kalamazoo, MI (US); Daniel Paul Uccello, Colchester, CT (US); Usa Reilly, West Haven, CT (US); Tamim Fehme Braish, Groton, CT (US)

(21) Appl. No.: 16/980,473

(22) PCT Filed: Mar. 14, 2019
(86) PCT No.: PCT/US2019/022170

§ 371 Date: Sep. 14, 2020

Related U.S. Application Data
(60) Provisional application No. 62/643,286, filed on Mar. 15, 2018.

(51) Int. Cl. C07F 9/6558; C07F 9/58; C07F 5/04; A61P 31/04

(52) U.S. Cl. CPC C07F 9/65583; A61P 31/04; C07F 5/04; C07F 9/58

(57) ABSTRACT

The present invention is directed to new pyridinone or pyrimidinone hydroxamic acid phosphates of Formula (1) and boronates of Formula (2), stereoisomers thereof;

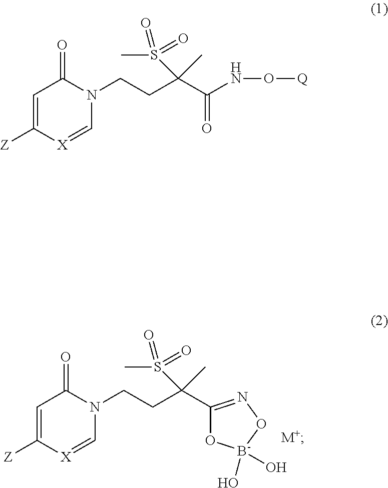

wherein Q is selected from the group consisting of —P(O)(OH)₂, —P(O)(OH)(O⁻M⁺), —P(O)(O⁻M⁺)₂ and —P(O)(O⁻)₂M²⁺; M⁺ at each occurrence is a pharmaceutically acceptable monovalent cation; and M²⁺ is a pharmaceutically acceptable divalent cation; X is CH or N; and Z is as defined herein; and their use as LpxC inhibitors and, more specifically, their use to treat bacterial infections.

### FIELD OF THE INVENTION

This invention relates to novel pyridinone and pyrimidinone hydroxamic acid phosphates and boronates. The invention also relates to methods of using such compounds in the treatment of bacterial infections (especially Gram-negative infections) and to pharmaceutical compositions containing such compounds.

### BACKGROUND OF THE INVENTION

Infection by Gram-negative bacteria such as Pseudomonas aeruginosa, Extended Spectrum β-lactamase producing (ESBL) Enterobacteriaceae, and Acinetobacter baumannii is a major health problem, especially in the case of hospital-acquired infections. In addition, there is an increasing level of resistance to current antibiotic therapies, which severely limits treatment options. For example, in 2002, 33% of Pseudomonas aeruginosa infections from intensive care units were resistant to fluoroquinolones, while resistance to imipenem was 22% (CID 42: 657-68, 2006). In addition, multi-drug resistant (MDR) infections are also increasing; in the case of Pseudomonas aeruginosa, MDR increased from 4% in 1992 to 14% in 2002 (Biochem Pharm 71: 991, 2006).

Gram-negative bacteria are unique in that their outer membrane contains lipopolysaccharide (LPS), which is crucial for maintaining membrane integrity, and is essential for bacterial viability (reviewed in Ann. Rev. Biochem 76: 295-329, 2007). The major lipid component of LPS is Lipid A, and inhibition of Lipid A biosynthesis is lethal to bacteria. Lipid A is synthesized on the cytoplasmic surface of the bacterial inner membrane via a pathway that consists of nine different enzymes. These enzymes are highly conserved in most Gram-negative bacteria. LpxC [UDP-3-O—(R-3-hydroxymyristoyl)-N-acetylglucosamine deacetylase] is the enzyme that catalyzes the first committed step in the Lipid A biosynthetic pathway, the removal of the N-acetyl group of UDP-3-O—(R-3-hydroxymyristoyl)-N-acetylglucosamine. LpxC is a Zn²⁺-dependent enzyme that has no mammalian homologue, making it a good target for the development of novel antibiotics. Several inhibitors of LpxC with low nM affinity have been reported (Biochemistry 45: 7940-48, 2006).

### SUMMARY OF THE INVENTION

The present invention is directed to certain novel pyridinone and pyrimidinone hydroxamic acid phosphates and boronates, pharmaceutical compositions comprising those compounds and methods of inhibiting LpxC and treating bacterial infections with those compounds.

In one embodiment of the present invention is a new pyridinone or pyrimidinone hydroxamic acid phosphate LpxC inhibitor compound of Formula (1), stereoisomers thereof,

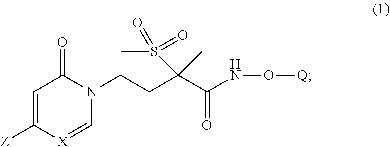

wherein Q is selected from the group consisting of —P(O)(OH)₂, —P(O)(OH)(O⁻M⁺), —P(O)(O⁻M⁺)₂ and —P(O)(O⁻)₂M²⁺;
X is CH or N;
Z is selected from the group consisting of

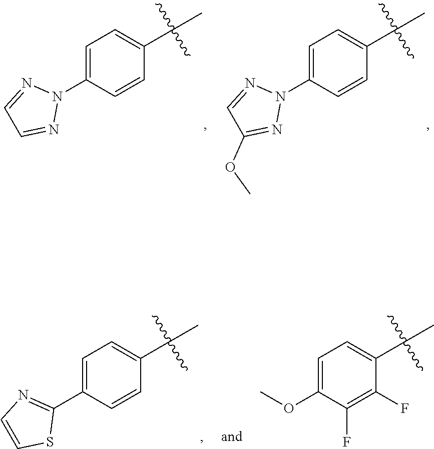 [SMILES: *c1ccc(-n2nccn2)cc1 ; *c1ccc(-n2ncc(OC)n2)cc1 ; *c1ccc(-c2nccs2)cc1 ; *c1ccc(OC)c(F)c1F]

M⁺ at each occurrence is a pharmaceutically acceptable monovalent cation; and
M²⁺ is a pharmaceutically acceptable divalent cation.

In another embodiment of the present invention is a Formula (1a) compound,

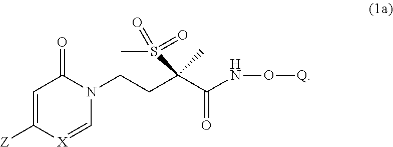

wherein Q is selected from the group consisting of —P(O)(OH)₂, —P(O)(OH)(O⁻M⁺), —P(O)(O⁻M⁺)₂ and —P(O)(O⁻)₂M²⁺;
X is CH or N;
Z is selected from the group consisting of

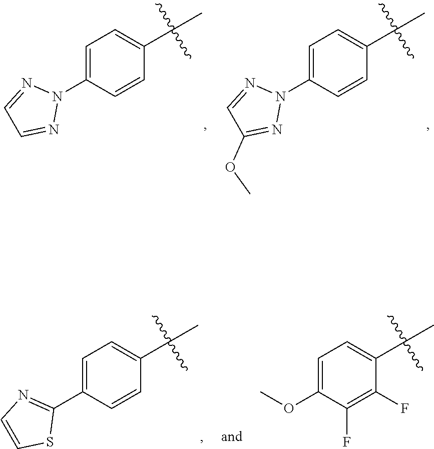 [SMILES: *c1ccc(-n2nccn2)cc1 ; *c1ccc(-n2ncc(OC)n2)cc1 ; *c1ccc(-c2nccs2)cc1 ; *c1ccc(OC)c(F)c1F]

M⁺ at each occurrence is a pharmaceutically acceptable monovalent cation; and
M²⁺ is a pharmaceutically acceptable divalent cation.

In another embodiment of the present invention is a Formula (1a) compound wherein X is CH; Z is

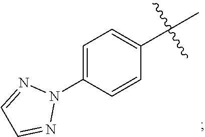 [SMILES: *c1ccc(-n2nccn2)cc1]

Q is selected from the group consisting of —P(O)(OH)₂, —P(O)(OH)(O⁻M⁺), —P(O)(O⁻M⁺)₂ and —P(O)(O⁻)₂M²⁺; M⁺ at each occurrence is a pharmaceutically acceptable monovalent cation; and M²⁺ is a pharmaceutically acceptable divalent cation.

In yet another embodiment of the present invention, is a Formula (1a) compound, wherein X is CH; Z is

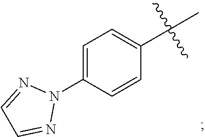 [SMILES: *c1ccc(-n2nccn2)cc1]

Q is —P(O)(OH)₂; —P(O)(OH)(O⁻M⁺); —P(O)(O⁻M⁺)₂; or —P(O)(O⁻)₂M²⁺; and M⁺ at each occurrence is independently selected from the group consisting of Li⁺, K⁺, Na⁺, NH₄⁺, NH₃⁺C(CH₂OH)₃, NH₂⁺(CH₂CH₃)₂, NH₂⁺(CH₂CH₃)₂, pyrrolidinium, and glycinium; and wherein M²⁺ is selected from the group consisting of Ca²⁺, Mg²⁺, and Zn²⁺. In another embodiment, M⁺ at each occurrence is independently selected from the group consisting of Li⁺, K⁺, and Na⁺; or M⁺ at each occurrence is a pharmaceutically acceptable monovalent cation independently selected from NH₄⁺, NH₃⁺C(CH₂OH)₃, NH₂⁺(CH₂CH₃)₂, NH₂⁺(CH₂CH₃)₂, pyrrolidinium, and glycinium; and wherein M²⁺ is selected from the group consisting of Ca²⁺, Mg²⁺, and Zn²⁺.

In yet another embodiment of the present invention, is Formula (1a) compound selected from the group consisting of:
(R)-4-(4-(4-(2H-1,2,3-triazol-2-yl)phenyl)-2-oxopyridin-1(2H)-yl)-2-methyl-2-(methylsulfonyl)butanamido phosphate, disodium salt;
(R)-4-(4-(4-(2H-1,2,3-triazol-2-yl)phenyl)-2-oxopyridin-1(2H)-yl)-2-methyl-2-(methylsulfonyl)butanamido phosphate, diammonium salt;
(R)-4-(4-(4-(2H-1,2,3-triazol-2-yl)phenyl)-2-oxopyridin-1(2H)-yl)-2-methyl-2-(methylsulfonyl)butanamido phosphate, dipotassium salt;
(R)-4-(4-(4-(2H-1,2,3-triazol-2-yl)phenyl)-2-oxopyridin-1(2H)-yl)-2-methyl-2-(methylsulfonyl)butanamido phosphate, dilithium salt;
(R)-4-(4-(4-(2H-1,2,3-triazol-2-yl)phenyl)-2-oxopyridin-1(2H)-yl)-2-methyl-2-(methylsulfonyl)butanamido phosphate, calcium salt;
(R)-4-(4-(4-(2H-1,2,3-triazol-2-yl)phenyl)-2-oxopyridin-1(2H)-yl)-2-methyl-2-(methylsulfonyl)butanamido phosphate, magnesium salt;
(R)-4-(4-(4-(2H-1,2,3-triazol-2-yl)phenyl)-2-oxopyridin-1(2H)-yl)-2-methyl-2-(methylsulfonyl)butanamido phosphate, zinc salt;
(R)-4-(4-(4-(2H-1,2,3-triazol-2-yl)phenyl)-2-oxopyridin-1(2H)-yl)-2-methyl-2-(methylsulfonyl)butanamido phosphate, pyrrolidine salt;
(R)-4-(4-(4-(2H-1,2,3-triazol-2-yl)phenyl)-2-oxopyridin-1(2H)-yl)-2-methyl-2-(methylsulfonyl)butanamido phosphate, tris-(hydroxymethyl)methylamine salt;
(R)-4-(4-(4-(2H-1,2,3-triazol-2-yl)phenyl)-2-oxopyridin-1(2H)-yl)-2-methyl-2-(methylsulfonyl)butanamido phosphate, diethylamine salt; and
(R)-4-(4-(4-(2H-1,2,3-triazol-2-yl)phenyl)-2-oxopyridin-1(2H)-yl)-2-methyl-2-(methylsulfonyl)butanamido phosphate, glycine salt, and other pharmaceutically acceptable salts thereof.

In another embodiment of the present invention is a Formula (1a) compound wherein X is N; Z is

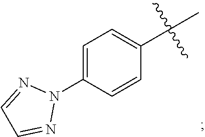 [SMILES: *c1ccc(-n2nccn2)cc1]

Q is selected from the group consisting of —P(O)(OH)₂, —P(O)(OH)(O⁻M⁺), —P(O)(O⁻M⁺)₂ and —P(O)(O⁻)₂M²⁺; M⁺ at each occurrence is a pharmaceutically acceptable monovalent cation; and M²⁺ is a pharmaceutically acceptable divalent cation.

In yet another embodiment of the present invention, is a Formula (1a) compound, wherein X is N; Z is

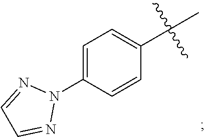 [SMILES: *c1ccc(-n2nccn2)cc1]

Q is —P(O)(OH)₂; —P(O)(OH)(O⁻M⁺); —P(O)(O⁻M⁺)₂; or —P(O)(O⁻)₂M²⁺; M⁺ at each occurrence is independently selected from the group consisting of Li⁺, K⁺, and Na⁺, or M⁺ at each occurrence is a pharmaceutically acceptable monovalent cation independently selected from NH⁴⁺, NH₃⁺C(CH₂OH)₃, NH₂⁺(CH₂CH₃)₂, NH₂⁺(CH₂CH₃)₂, pyrrolidinium, and glycinium; and wherein M²⁺ is selected from the group consisting of Ca²⁺, Mg²⁺, and Zn²⁺.

In yet another embodiment of the present invention, are boronate prodrugs of Formula (1) and Formula (1a) that are compounds of Formula (2) and Formula (2a), respectively,

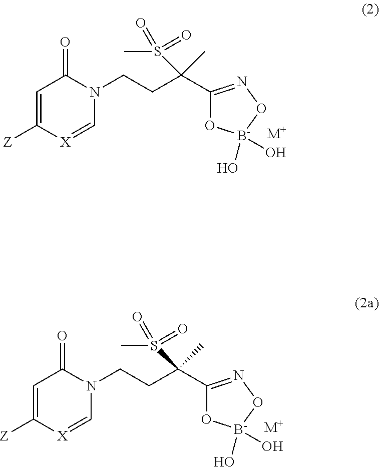

wherein X is CH or N; and Z is selected from the group consisting of

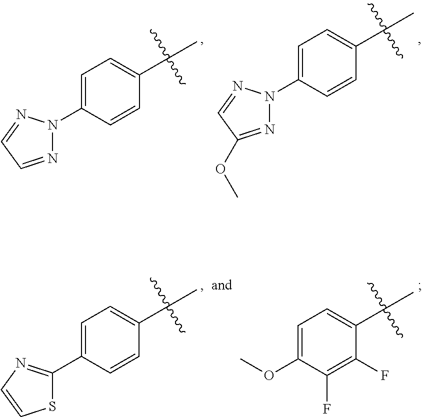 [SMILES: *c1ccc(-n2nccn2)cc1 ; *c1ccc(-n2ncc(OC)n2)cc1 ; *c1ccc(-c2nccs2)cc1 ; *c1ccc(OC)c(F)c1F]

and M⁺ is a pharmaceutically acceptable monovalent cation.

In yet another embodiment of the present invention is a Formula (2a) compound wherein X is CH; Z is

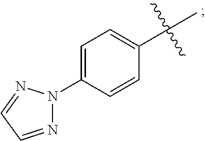 [SMILES: *c1ccc(-n2nccn2)cc1]

M⁺ is a pharmaceutically acceptable monovalent cation selected from the group consisting of Li⁺, K⁺, and Na⁺; or M⁺ is a pharmaceutically acceptable monovalent cation independently selected from NH⁴⁺, NH₃⁺C(CH₂OH)₃, NH₂⁺(CH₂CH₃)₂; NH₂⁺(CH₂CH₃)₂; pyrrolidinium; and glycinium.

In yet another embodiment of the present invention is a Formula (2a) compound wherein X is N; Z is

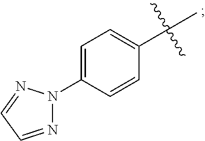

wherein M⁺ is a pharmaceutically acceptable monovalent cation selected from the group consisting of Li⁺, K⁺, and Na⁺; or M⁺ is a pharmaceutically acceptable monovalent cation independently selected from NH⁴⁺, NH₃⁺C(CH₂OH)₃, NH₂⁺(CH₂CH₃)₂; NH₂⁺(CH₂CH₃)₂; pyrrolidinium; and glycinium.

In yet another embodiment of the present invention is a Formula (2a) compound that is a boronate prodrug of (R)-4-(4-(4-(2H-1,2,3-triazol-2-yl)phenyl)-2-oxopyridin-1(2H)-yl)-N-hydroxy-2-methyl-2-(methylsulfonyl) butanamide, and pharmaceutically acceptable salts thereof. In yet another embodiment of the present invention is a Formula (2a) compound that is sodium (R)-5-(4-(4-(4-(2H-1,2,3-triazol-2-yl)phenyl)-2-oxopyridin-1(2H)-yl)-2-(methylsulfonyl)butan-2-yl)-2,2-dihydroxy-1,3,4,2-dioxazaborol-2-uide, and other pharmaceutically acceptable salts thereof.

In yet another embodiment of the present invention is a Formula (1a) compound selected from the group consisting of:
(2R)-4-[4-(2,3-difluoro-4-methoxyphenyl)-2-oxopyridin-1(2H)-yl]-N-hydroxy-2-methyl-2-(methylsulfonyl)butanamido phosphate, disodium salt;
(R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate, diammonium salt;
(2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, disodium salt;
(2R)—N-hydroxy-2-methyl-2-(methylsulfonyl)-4-{2-oxo-4-[4-(1,3-thiazol-2-yl)phenyl]pyridin-1(2H)-yl}butanamido phosphate, disodium salt;
(R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate, disodium salt;
(2R)-4-[4-(2,3-difluoro-4-methoxyphenyl)-2-oxopyridin-1(2H)-yl]-N-hydroxy-2-methyl-2-(methylsulfonyl)butanamido phosphate, diammonium salt;
(2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, diammonium salt;
(2R)—N-hydroxy-2-methyl-2-(methylsulfonyl)-4-{2-oxo-4-[4-(1,3-thiazol-2-yl)phenyl]pyridin-1(2H)-yl}butanamido phosphate, ammonium salt;
(2R)-4-[4-(2,3-difluoro-4-methoxyphenyl)-2-oxopyridin-1(2H)-yl]-N-hydroxy-2-methyl-2-(methylsulfonyl)butanamido phosphate, dipotassium salt;
(2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, dipotassium salt;
(2R)—N-hydroxy-2-methyl-2-(methylsulfonyl)-4-{2-oxo-4-[4-(1,3-thiazol-2-yl)phenyl]pyridin-1(2H)-yl}butanamido phosphate, dipotassium salt;
(R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate, dipotassium salt;
(2R)-4-[4-(2,3-difluoro-4-methoxyphenyl)-2-oxopyridin-1(2H)-yl]-N-hydroxy-2-methyl-2-(methylsulfonyl)butanamido phosphate, dilithium salt;
(2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, dilithium salt;
(2R)—N-hydroxy-2-methyl-2-(methylsulfonyl)-4-{2-oxo-4-[4-(1,3-thiazol-2-yl)phenyl]pyridin-1(2H)-yl}butanamido phosphate, dilithium salt; and
(R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate, dilithium salt, and other pharmaceutically acceptable salts thereof.

In yet another embodiment of the present invention is a Formula (2a) compound selected from the group consisting of:
sodium (R)-5-(4-(4-(2,3-difluoro-4-methoxyphenyl)-2-oxopyridin-1(2H)-yl)-2-(methylsulfonyl)butan-2-yl)-2,2-dihydroxy-1,3,4,2-dioxazaborol-2-uide
sodium (R)-2,2-dihydroxy-5-(4-(4-(4-(4-methoxy-2H-1,2,3-triazol-2-yl)phenyl)-2-oxopyridin-1(2H)-yl)-2-(methylsulfonyl)butan-2-yl)-1,3,4,2-dioxazaborol-2-uide;
sodium (R)-2,2-dihydroxy-5-(2-(methylsulfonyl)-4-(2-oxo-4-(4-(thiazol-2-yl)phenyl)pyridin-1(2H)-yl)butan-2-yl)-1,3,4,2-dioxazaborol-2-uide; and
sodium (R)-5-(4-(4-(4-(2H-1,2,3-triazol-2-yl)phenyl)-6-oxopyrimidin-1(6H)-yl)-2-(methylsulfonyl)butan-2-yl)-2,2-dihydroxy-1,3,4,2-dioxazaborol-2-uide; and other pharmaceutically acceptable salts thereof.

In yet another embodiment of the present invention, is a pharmaceutical composition comprising a Formula (1), Formula (1a), Formula (2), or Formula (2a) compound in admixture with at least one pharmaceutically acceptable excipient, diluent or carrier.

In yet another embodiment of the present invention, is a pharmaceutical composition comprising a Formula (1), Formula (1a), Formula (2), or Formula (2a) compound, or pharmaceutically acceptable salt thereof, in admixture with at least one pharmaceutically acceptable excipient, diluent or carrier; for administration to a patient by oral, topical, or injectable administration.

In yet another embodiment of the present invention, is a method for treating a bacterial infection in a patient, the method comprising administering a therapeutically effective amount of a Formula (1), Formula (1a), Formula (2), or Formula (2a) compound, or pharmaceutically acceptable salt thereof, to a patient in need thereof. In yet another embodiment of the present invention, is a method for treating a bacterial infection in a patient, the method comprising administering a therapeutically effective amount of a Formula (1), Formula (1a), Formula (2), or Formula (2a) compound, or pharmaceutically acceptable salt thereof, to a patient in need thereof, by oral, topical, or injectable administration.

In yet another embodiment of the present invention, is the use of a Formula (1), Formula (1a), Formula (2), or Formula (2a) compound, or pharmaceutically acceptable salt thereof, for preparing a medicament for treating a bacterial infection in a patient.

In yet another embodiment, the bacterial infection is a Gram-negative bacterial infection. In yet another embodiment, the Gram-negative bacterial infection is caused by a Gram-negative bacteria selected from the group consisting of Mannheimia haemolytica, Pasteurella multocida, Histophilus somni, Actinobacillus pleuropneumoniae, Salmonella enteritidis, Salmonella gallinarium, Lawsonia intracellularis, Brachyspira hyodysenteriae, Brachyspira pilosicoli, Acinetobacter baumannii, Acinetobacter spp., Citrobacter spp., Enterobacter aerogenes, Enterobacter cloacae, Escherichia coli, Klebsiella oxytoca, Klebsiella pneumoniae, Serratia marcescens, Stenotrophomonas maltophilia, and Pseudomonas aeruginosa. In yet another embodiment, the Gram-negative bacterial infection is selected from the group consisting of respiratory infection, gastrointestinal infection, nosocomial pneumonia, urinary tract infection, bacteremia, sepsis, skin infection, soft-tissue infection, intraabdominal infection, lung infection, endocarditis, diabetic foot infection, osteomyelitis and central nervous system infection.

### DETAILED DESCRIPTION OF THE INVENTION

### Definitions

As used throughout this application, including the claims, the following terms have the meanings defined below, unless specifically indicated otherwise. The plural and singular should be treated as interchangeable, other than the indication of number:

“alkyl” refers to a linear or branched-chain hydrocarbyl substituent (i.e., a substituent obtained from a hydrocarbon by removal of a hydrogen); in one embodiment containing from one (C₁) to twelve (C₁₂) carbon atoms, i.e., C₁-C₁₂. Non-limiting examples of such substituents include methyl, ethyl (C₂), propyl (including n-propyl and isopropyl), butyl (including n-butyl, isobutyl, sec-butyl and tert-butyl), pentyl, isoamyl, hexyl, heptyl, octyl, nonyl, decyl, undecyl, dodecyl and the like.

“cycloalkyl” refers to a carbocyclic substituent obtained by removing a hydrogen from a saturated carbocyclic molecule, for example one having three to six carbon atoms. The term “C₃₋₆cycloalkyl” means a radical of a three to six membered ring which includes the groups cyclopropyl, cyclobutyl, cyclopentyl and cyclohexyl.

In some instances, the number of carbon atoms in a hydrocarbyl substituent (i.e., alkyl, cycloalkyl, etc.) is indicated by the prefix “Cₓ-C_{y}-” or “C_{x-y}”, wherein x is the minimum and y is the maximum number of carbon atoms in the substituent. Thus, for example, “C₁-C₁₂-alkyl” or “C₁₋₁₂ alkyl” refers to an alkyl substituent containing from 1 to 12 carbon atoms and “C₁-C₆-alkyl” or “C₁₋₆ alkyl” refers to an alkyl substituent containing from 1 to 6 carbon atoms. Illustrating further, C₃-C₆cycloalkyl or C₃₋₆-cycloalkyl refers to saturated cycloalkyl group containing from 3 to 6 carbon ring atoms.

“compounds of the present invention”, means Formula (1), Formula (1a), Formula (2), and Formula (2a) compounds, stereoisomers thereof, and pharmaceutically acceptable salts thereof.

“divalent cation”, defined by M²⁺ herein, is a cation with a valence of 2, and includes the metal cations: Mg²⁺, Ca²⁺, and Zn²⁺.

“geometric isomer” means any of two or more stereoisomers that differ in the arrangement of atoms or groups of atoms around a structurally rigid bond, such as a double bond or a ring and are defined as cis (same side) and trans (opposite side) of the bond or ring.

“isomer” means “stereoisomer” and “geometric isomer” as defined herein.

“monovalent cation”, defined by M⁺ herein, includes ammonium (NH₄₊), mono-, di-, tri- and tetra-(C₁-C₁₂alkyl)ammonium (i.e. (C₁-C₁₂alkyl)NH₃⁺, (C₁-C₁₂alkyl)₂NH₂⁺, (C₁-C₁₂alkyl)₃NH⁺, and (C₁-C₁₂alkyl)₄N⁺) wherein the alkyl group(s) may be substituted as specified, mono-, di-, tri- and tetra-(C₃-C₆cycloalkyl)ammonium (i.e. (C₃-C₆cycloalkyl)NH₃⁺, (C₃-C₆cycloalkyl)₂NH₂⁺, (C₃-C₆cycloalkyl)₃NH⁺, and (C₃-C₆cycloalkyl)₄N⁺), alkali metal ions such as sodium, lithium and potassium ions, ions of organic amines such as pyrrolidine, piperidine or pyridine and ions of amino acids such as ions of glycine, alanine, 3-alanine, valine, lysine, isoleucine, leucine, methionine, threonine, asparagine, glutamine, histidine, arginine, ornithine, tryptophane, proline, glutamine, cysteine, phenylalanine, tyrosine and serine. When the organic amine or amino acid is in its protonated form this can be denoted by the use of the suffix “ium”. For example, protonated pyrrolidine is pyrrolidinium, protonated piperidine is piperidinium, protonated pyridine is pyridinium and protonated glycine is glycinium.

“parent compound” refers to the biologically active entity that is released via enzymatic action of a metabolic or catabolic process, or via a chemical process following administration of the phosphate salt from the Formula (1) or Formula (1a) compounds or the boronate of the Formula (2) or Formula (2a) compounds.

“patient” refers to warm blooded animals such as for example, humans and non-humans. The term non-humans refer to animals such as livestock (i.e., cattle, swine, sheep, and goats), and companion animals (i.e., cat, dog, and horse); and also includes other non-human animals, e.g., guinea pigs, mice, rats, gerbils, rabbits, monkeys, chimpanzees, and the like.

“pharmaceutically acceptable” indicates that the substance or composition must be compatible chemically and/or toxicologically, with the other ingredients comprising a formulation, and/or the patient being treated therewith. The term is synonymous to veterinary acceptable (i.e., ingredients are compatible with a non-human patient).

“prodrug” refers to compounds which are drug precursors which, following administration and absorption, release the drug in vivo via some metabolic, catabolic or chemical process; for example, by hydrolytic cleavage of the phosphate in the Formula (1) and Formula (1a) compounds or of the boronate in Formula (2) and Formula (2a) compounds.

“pyridone” and “pyridinone” have been used interchangeably within this application.

No difference or distinction is meant, unless otherwise noted.

“stereoisomer” means compounds that possess one or more chiral centers and each center may exist in the R or S configuration. Stereoisomers include all diastereomeric, enantiomeric and epimeric forms as well as racemates and mixtures thereof.

“therapeutically effective amount” refers to an amount of a compound of the invention (i.e., a compound of Formula I, Ia, II, or IIa) that, when administered to a patient, provides the desired effect; e.g., lessening in the severity of the symptoms associated with a bacterial infection, decreasing the number of bacteria in the affected tissue, and/or preventing bacteria in the affected tissue from increasing in number (localized or systemic).

“treat”, “treating”, ‘treatment”, and the like refers to the ability of the compounds of the present invention to relieve, alleviate or slow the progression of the patient's bacterial infection (or condition) or any tissue damage associated with the disease.

Compounds of the present invention are LpxC inhibitors that are useful for treating patients with a bacterial infection caused by Gram-negative bacteria.

A first embodiment of a first aspect of the present invention is a new pyridinone or pyrimidinone hydroxamic acid phosphate LpxC inhibitor Formula (1) compound,

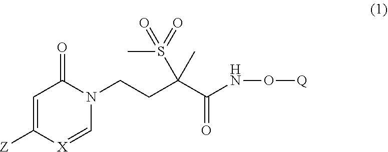

or a pharmaceutically acceptable salt thereof; stereoisomers thereof, and pharmaceutically acceptable salts thereof; wherein Q is selected from the group consisting of —P(O)(OH)₂, —P(O)(OH)(O⁻M⁺), —P(O)(O⁻M⁺)₂ and —P(O)(O⁻)₂M²⁺; X is CH or N; and wherein Z is selected from the group consisting of

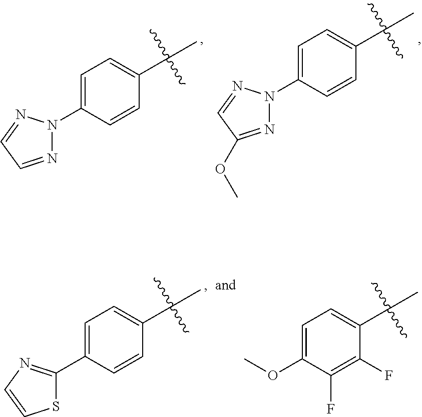 [SMILES: *c1ccc(-n2nccn2)cc1 ; *c1ccc(-n2ncc(OC)n2)cc1 ; *c1ccc(-c2nccs2)cc1 ; *c1ccc(OC)c(F)c1F]

M⁺ at each occurrence is a pharmaceutically acceptable monovalent cation; and M²⁺ is a pharmaceutically acceptable divalent cation.

A first embodiment of a second aspect of the present invention is the new boronate Lpxc inhibitor compound of Formula (2)

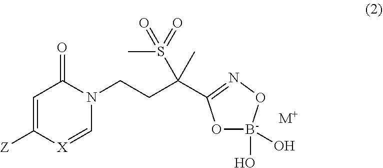

wherein X is CH or N; M⁺ is a pharmaceutically acceptable monovalent cation; and Z is selected from the group consisting of

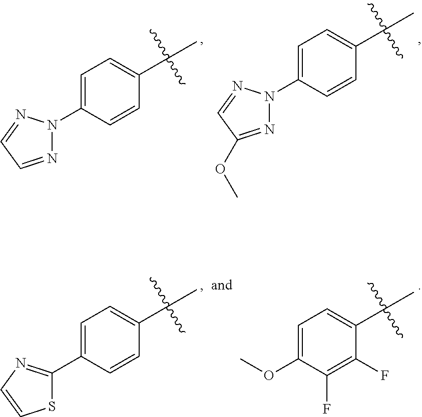 [SMILES: *c1ccc(-n2nccn2)cc1 ; *c1ccc(-n2ncc(OC)n2)cc1 ; *c1ccc(-c2nccs2)cc1 ; *c1ccc(OC)c(F)c1F]

The compounds of Formula (1) and Formula (2) once administered to a patient in need thereof exhibit antibacterial activity, especially against Gram-negative organisms. These compounds may be used to treat bacterial infections in mammals, especially humans. The compounds may also be used for veterinary applications, such as treating infections in livestock and companion animals.

The compounds of Formula (1) and Formula (2) are useful for treating a variety of infections; especially Gram-negative infections including nosocomial pneumonia, urinary tract infections, systemic infections (bacteremia and sepsis), skin and soft tissue infections, surgical infections, intraabdominal infections, lung infections (including those in patients with cystic fibrosis), Helicobacter pylori (and relief of associated gastric complications such as peptic ulcer disease, gastric carcinogenesis, etc.), endocarditis, diabetic foot infections, osteomyelitis, and central nervous system infections.

In order to simplify administration, the compounds will typically be admixed with at least one excipient and formulated into a pharmaceutical dosage form. Examples of such dosage forms include tablets, capsules, solutions/suspensions for injection, aerosols for inhalation, cream/ointments for topical, otic or ophthalmic use, solutions/suspensions for oral ingestion, and as medicated feed additives. The instant compounds possess enhanced aqueous solubility compared to the parent hydroxamic acid compound from which they are derived and therefore the instant compounds can advantageously be employed in injectable dosage forms.

A second embodiment of the first aspect of the present invention is the compound of the first embodiment of the first aspect of Formula 1a

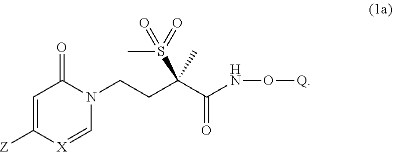

A third embodiment of the first aspect of the present invention is the compound of the second embodiment of the first aspect wherein X is CH.

A fourth embodiment of the first aspect of the present invention is the compound of the third embodiment of the first aspect wherein Z is

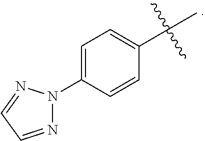 [SMILES: *c1ccc(-n2nccn2)cc1]

A fifth embodiment of the first aspect of the present invention is the compound of the third embodiment of the first aspect wherein Z is

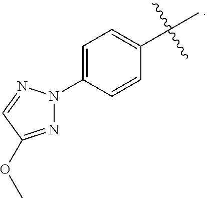 [SMILES: *c1ccc(-n2ncc(OC)n2)cc1]

A sixth embodiment of the first aspect of the present invention is the compound of the third embodiment of the first aspect wherein Z is

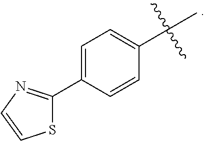 [SMILES: *c1ccc(-c2nccs2)cc1]

A seventh embodiment of a first aspect of the present invention is the compound of the third embodiment of the first aspect wherein Z is

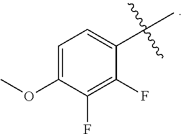 [SMILES: *c1ccc(OC)c(F)c1F]

An eighth embodiment of a first aspect of the present invention is the compound of the second embodiment of the first aspect wherein X is N; and Z is

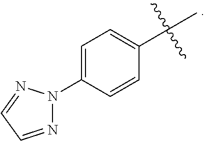 [SMILES: *c1ccc(-n2nccn2)cc1]

A ninth embodiment of a first aspect of the present invention is the compound of the second embodiment of the first aspect wherein Q is —P(O)(OH)₂. A tenth embodiment of a first aspect of the present invention is the compound of the second embodiment of the first aspect wherein Q is —P(O)(OH)(O⁻M⁺) or —P(O)(O⁻M⁺)₂. An eleventh embodiment of a first aspect of the present invention is the compound of the tenth embodiment of the first aspect wherein Q is —P(O)(O⁻M⁺)₂. A twelfth embodiment of a first aspect of the present invention is the compound of the second embodiment of the first aspect wherein Q is —P(O)(O⁻)₂M²⁺. A thirteenth embodiment of a first aspect of the present invention is the compound of the tenth embodiment of the first aspect wherein M⁺ at each occurrence is independently selected from the group consisting of Li⁺, K⁺ and Na⁺.

A fourteenth embodiment of a first aspect of the present invention is the compound of the tenth embodiment of the first aspect wherein M⁺ at each occurrence is a pharmaceutically acceptable monovalent cation independently selected from ammonium, (C₁-C₁₂alkyl)ammonium, (C₁-C₁₂alkyl)₂ammonium, (C₁-C₁₂alkyl)₃ammonium, (C₁-C₁₂alkyl)₄ammonium, (C₃-C₆cycloalkyl)ammonium, (C₃-C₆cycloalkyl)₂ammonium, (C₃-C₆cycloalkyl)₃ammonium, (C₃-C₆cycloalkyl)₄ammonium, pyrrolidinium, piperidinium and pyridinium; wherein each of the (C₁-C₁₂alkyl) or (C₃-C₆cycloalkyl) moieties are optionally substituted with one to three hydroxy or halo.

A fifteenth embodiment of a first aspect of the present invention is the compound of the tenth embodiment of the first aspect wherein M⁺ at each occurrence is a pharmaceutically acceptable monovalent cation independently selected from the group consisting of glycinium, alaninium, β-alaninium, valinium, lysinium, isoleucinium, leucinium, methioninium, threoninium, asparaginium, glutaminium, histidinium, argininium, ornithinium, tryptophanium, prolinium, glutaminium, cysteinium, phenylalaninium, tyrosinium and serinium.

A sixteenth embodiment of a first aspect of the present invention is the compound of the tenth embodiment of the first aspect wherein M⁺ is Na⁺. A seventeenth embodiment of a first aspect of the present invention is the compound of the tenth embodiment of the first aspect wherein M⁺ is K⁺. An eighteenth embodiment of a first aspect of the present invention is the compound of the tenth embodiment of the first aspect wherein M⁺ is Li⁺.

A nineteenth embodiment of a first aspect of the present invention is the compound of the tenth embodiment of the first aspect wherein M⁺ is NH₄⁺. A twentieth embodiment of a first aspect of the present invention is the compound of the tenth embodiment of the first aspect wherein M⁺ is NH₃⁺C(CH₂OH)₃. A twentyfirst embodiment of a first aspect of the present invention is the compound of the tenth embodiment of the first aspect wherein wherein M⁺ is NH₂⁺(CH₂CH₃)₂. A twentysecond embodiment of a first aspect of the present invention is the compound of the twelfth embodiment of the first aspect wherein M²⁺ is selected from the group consisting of Ca²⁺, Mg²⁺ and Zn²⁺.

A twentythird embodiment of a first aspect of the present invention is a compound of the third embodiment of the first aspect selected from the group consisting of:
(R)-4-(4-(4-(2H-1,2,3-triazol-2-yl)phenyl)-2-oxopyridin-1(2H)-yl)-2-methyl-2-(methylsulfonyl)butanamido phosphate, disodium salt;
(R)-4-(4-(4-(2H-1,2,3-triazol-2-yl)phenyl)-2-oxopyridin-1(2H)-yl)-2-methyl-2-(methylsulfonyl)butanamido phosphate, diammonium salt;
(R)-4-(4-(4-(2H-1,2,3-triazol-2-yl)phenyl)-2-oxopyridin-1(2H)-yl)-2-methyl-2-(methylsulfonyl)butanamido phosphate, dipotassium salt;
(R)-4-(4-(4-(2H-1,2,3-triazol-2-yl)phenyl)-2-oxopyridin-1(2H)-yl)-2-methyl-2-(methylsulfonyl)butanamido phosphate, dilithium salt;
(R)-4-(4-(4-(2H-1,2,3-triazol-2-yl)phenyl)-2-oxopyridin-1(2H)-yl)-2-methyl-2-(methylsulfonyl)butanamido phosphate, calcium salt;
(R)-4-(4-(4-(2H-1,2,3-triazol-2-yl)phenyl)-2-oxopyridin-1(2H)-yl)-2-methyl-2-(methylsulfonyl)butanamido phosphate, magnesium salt;
(R)-4-(4-(4-(2H-1,2,3-triazol-2-yl)phenyl)-2-oxopyridin-1(2H)-yl)-2-methyl-2-(methylsulfonyl)butanamido phosphate, zinc salt;
(R)-4-(4-(4-(2H-1,2,3-triazol-2-yl)phenyl)-2-oxopyridin-1(2H)-yl)-2-methyl-2-(methylsulfonyl)butanamido phosphate, pyrrolidine salt;
(R)-4-(4-(4-(2H-1,2,3-triazol-2-yl)phenyl)-2-oxopyridin-1(2H)-yl)-2-methyl-2-(methylsulfonyl)butanamido phosphate, tris-(hydroxymethyl)methylamine salt;
(R)-4-(4-(4-(2H-1,2,3-triazol-2-yl)phenyl)-2-oxopyridin-1(2H)-yl)-2-methyl-2-(methylsulfonyl)butanamido phosphate, diethylamine salt; and
(R)-4-(4-(4-(2H-1,2,3-triazol-2-yl)phenyl)-2-oxopyridin-1(2H)-yl)-2-methyl-2-(methylsulfonyl)butanamido phosphate, glycine salt, and other pharmaceutically acceptable salts thereof.

A twentyfourth embodiment of a first aspect of the present invention is a compound of the second embodiment of the first aspect selected from the group consisting of:
(2R)-4-[4-(2,3-difluoro-4-methoxyphenyl)-2-oxopyridin-1(2H)-yl]-N-hydroxy-2-methyl-2-(methylsulfonyl)butanamido phosphate, disodium salt;
(R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate, diammonium salt;
(2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, disodium salt;
(2R)—N-hydroxy-2-methyl-2-(methylsulfonyl)-4-{2-oxo-4-[4-(1,3-thiazol-2-yl)phenyl]pyridin-1(2H)-yl}butanamido phosphate, disodium salt;
(R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate, disodium salt;
(2R)-4-[4-(2,3-difluoro-4-methoxyphenyl)-2-oxopyridin-1(2H)-yl]-N-hydroxy-2-methyl-2-(methylsulfonyl)butanamido phosphate, diammonium salt;
(2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, diammonium salt;
(2R)—N-hydroxy-2-methyl-2-(methylsulfonyl)-4-{2-oxo-4-[4-(1,3-thiazol-2-yl)phenyl]pyridin-1(2H)-yl}butanamido phosphate, ammonium salt;
(2R)-4-[4-(2,3-difluoro-4-methoxyphenyl)-2-oxopyridin-1(2H)-yl]-N-hydroxy-2-methyl-2-(methylsulfonyl)butanamido phosphate, dipotassium salt;
(2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, dipotassium salt;
(2R)—N-hydroxy-2-methyl-2-(methylsulfonyl)-4-{2-oxo-4-[4-(1,3-thiazol-2-yl)phenyl]pyridin-1(2H)-yl}butanamido phosphate, dipotassium salt;
(R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate, dipotassium salt;
(2R)-4-[4-(2,3-difluoro-4-methoxyphenyl)-2-oxopyridin-1(2H)-yl]-N-hydroxy-2-methyl-2-(methylsulfonyl)butanamido phosphate, dilithium salt;
(2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, dilithium salt;
(2R)—N-hydroxy-2-methyl-2-(methylsulfonyl)-4-{2-oxo-4-[4-(1,3-thiazol-2-yl)phenyl]pyridin-1(2H)-yl}butanamido phosphate, dilithium salt; and
(R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate, dilithium salt, and other pharmaceutically acceptable salts thereof.

A second embodiment of a second aspect of the present invention is the compound of the first embodiment of the second aspect of Formula (2a)

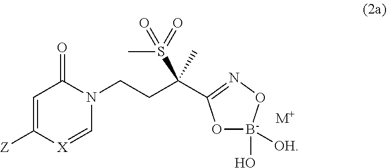

A third embodiment of a second aspect of the present invention is the compound of the second embodiment of the second aspect wherein X is CH.

A fourth embodiment of a second aspect of the present invention is the compound of the third embodiment of the second aspect wherein Z is

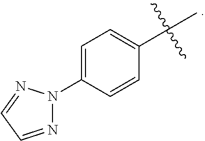 [SMILES: *c1ccc(-n2nccn2)cc1]

A fifth embodiment of a second aspect of the present invention is the compound of the third embodiment of the second aspect wherein Z is

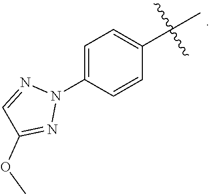 [SMILES: *c1ccc(-n2ncc(OC)n2)cc1]

A sixth embodiment of a second aspect of the present invention is the compound of the third embodiment of the second aspect wherein Z is

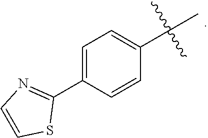 [SMILES: *c1ccc(-c2nccs2)cc1]

A seventh embodiment of a second aspect of the present invention is the compound of the third embodiment of the second aspect wherein Z is

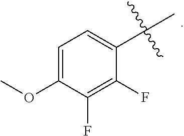 [SMILES: *c1ccc(OC)c(F)c1F]

An eighth embodiment of a second aspect of the present invention is the compound of the second embodiment of the second aspect wherein X is N; and Z is

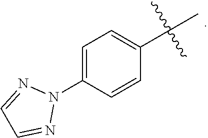 [SMILES: *c1ccc(-n2nccn2)cc1]

A ninth embodiment of a second aspect of the present invention is the compound of the second embodiment of the second aspect wherein M⁺ is selected from the group consisting of Li⁺, K⁺ and Na⁺.

A tenth embodiment of a second aspect of the present invention is the compound of the second embodiment of the second aspect wherein M⁺ is selected from the group consisting of ammonium, (C₁-C₁₂alkyl)ammonium, (C₁-C₁₂alkyl)₂ammonium, (C₁-C₁₂alkyl)₃ammonium, (C₁-C₁₂alkyl)₄ammonium, (C₃-C₆cycloalkyl)ammonium, (C₃-C₆cycloalkyl)₂ammonium, (C₃-C₆cycloalkyl)₃ammonium, (C₃-C₆cycloalkyl)₄ammonium, pyrrolidinium, piperidinium and pyridinium; wherein each of the (C₁-C₁₂alkyl) or (C₃-C₆cycloalkyl) moieties are optionally substituted with one to three hydroxy or halo.

An eleventh embodiment of a second aspect of the present invention is the compound of the second embodiment of the second aspect wherein M⁺ is selected from the group consisting of glycinium, alaninium, 3-alaninium, valinium, lysinium, isoleucinium, leucinium, methioninium, threoninium, asparaginium, glutaminium, histidinium, argininium, ornithinium, tryptophanium, prolinium, glutaminium, cysteinium, phenylalaninium, tyrosinium and serinium.

A twelfth embodiment of a second aspect of the present invention is the compound of the second embodiment of the second aspect wherein M⁺ is Na⁺. A thirteenth embodiment of a second aspect of the present invention is the compound of the second embodiment of the second aspect wherein M⁺ is K⁺. A fourteenth embodiment of a second aspect of the present invention is the compound of the second embodiment of the second aspect wherein M⁺ is Li⁺. A fifteenth embodiment of a second aspect of the present invention is the compound of the second embodiment of the second aspect wherein M⁺ is NH₄₊. A sixteenth embodiment of a second aspect of the present invention is the compound of the second embodiment of the second aspect wherein M⁺ is NH₃⁺C(CH₂OH)₃. A seventeenth embodiment of a second aspect of the present invention is the compound of the second embodiment of the second aspect wherein M+ is NH₂⁺(CH₂CH₃)₂.

An eighteenth embodiment of a second aspect of the present invention is the second embodiment of the second aspect that is a boronate prodrug of (R)-4-(4-(4-(2H-1,2,3-triazol-2-yl)phenyl)-2-oxopyridin-1(2H)-yl)-N-hydroxy-2-methyl-2-(methylsulfonyl) butanamide, and pharmaceutically acceptable salts thereof.

A nineteenth embodiment of a second aspect of the present invention is the second embodiment of the second aspect that is a boronate prodrug of (R)-4-(4-(4-(2H-1,2,3-triazol-2-yl)phenyl)-2-oxopyridin-1(2H)-yl)-N-hydroxy-2-methyl-2-(methylsulfonyl) butanamide, that is sodium (R)-5-(4-(4-(4-(2H-1,2,3-triazol-2-yl)phenyl)-2-oxopyridin-1(2H)-yl)-2-(methylsulfonyl)butan-2-yl)-2,2-dihydroxy-1,3,4,2-dioxazaborol-2-uide, and other pharmaceutically acceptable salts thereof.

A twentieth embodiment of a second aspect of the present invention is the second embodiment of the second aspect that is a boronate prodrug selected from the group consisting of:
sodium (R)-5-(4-(4-(2,3-difluoro-4-methoxyphenyl)-2-oxopyridin-1(2H)-yl)-2-(methylsulfonyl)butan-2-yl)-2,2-dihydroxy-1,3,4,2-dioxazaborol-2-uide
sodium (R)-2,2-dihydroxy-5-(4-(4-(4-(4-methoxy-2H-1,2,3-triazol-2-yl)phenyl)-2-oxopyridin-1(2H)-yl)-2-(methylsulfonyl)butan-2-yl)-1,3,4,2-dioxazaborol-2-uide;
sodium (R)-2,2-dihydroxy-5-(2-(methylsulfonyl)-4-(2-oxo-4-(4-(thiazol-2-yl)phenyl)pyridin-1(2H)-yl)butan-2-yl)-1,3,4,2-dioxazaborol-2-uide; and
sodium (R)-5-(4-(4-(4-(2H-1,2,3-triazol-2-yl)phenyl)-6-oxopyrimidin-1(6H)-yl)-2-(methylsulfonyl)butan-2-yl)-2,2-dihydroxy-1,3,4,2-dioxazaborol-2-uide; and other pharmaceutically acceptable salts thereof.

A first embodiment of a third aspect of the present invention is a pharmaceutical composition comprising a compound according to any one of the embodiments of the first or second aspects in admixture with at least one pharmaceutically acceptable excipient, diluent or carrier.

A first embodiment of a fourth aspect of the present invention is a method for treating a Gram-negative bacterial infection in a patient, the method comprising administering a therapeutically effective amount of a compound according to any one of the embodiments of the first or second aspects to a patient in need thereof.

A second embodiment of a fourth aspect of the present invention is the method of the first embodiment of the fourth aspect wherein the Gram-negative bacterial infection is caused by a Gram-negative bacteria selected from the group consisting of Mannheimia haemolytica, Pasteurella multocida, Histophilus somni, Actinobacillus pleuropneumoniae, Salmonella enteritidis, Salmonella gallinarium, Lawsonia intracellularis, Brachyspira hyodysenteriae, Brachyspira pilosicoli, Acinetobacter baumannii, Acinetobacter spp., Citrobacter spp., Enterobacter aerogenes, Enterobacter cloacae, Escherichia coli, Klebsiella oxytoca, Klebsiella pneumoniae, Serratia marcescens, Stenotrophomonas maltophilia, and Pseudomonas aeruginosa.

A third embodiment of a fourth aspect of the present invention is the method of the first embodiment of the fourth aspect wherein the Gram-negative bacterial infection is selected from the group consisting of respiratory infection, gastrointestinal infection, nosocomial pneumonia, urinary tract infection, bacteremia, sepsis, skin infection, soft-tissue infection, intraabdominal infection, lung infection, endocarditis, diabetic foot infection, osteomyelitis and central nervous system infection.

The invention relates to base addition salts of the compounds of the present invention. The chemical bases that may be used as reagents to prepare these pharmaceutically acceptable base salts are those that form non-toxic base salts with such compounds. Such non-toxic base salts include, but are not limited to those derived from such pharmacologically acceptable cations (M⁺ or M²⁺) such as alkali metal cations (e.g., lithium, potassium and sodium) and alkaline earth metal cations (e.g., calcium, magnesium and zinc), ammonium, alkylamine, dialkylamine, trialkylamine, tetralkylammonium, pyridinium or water-soluble amine addition salts such as N-methylglucamine-(meglumine), and the lower alkanolammonium and other base salts of pharmaceutically acceptable organic amines such as piperidine, N-methylpiperidine, morpholine, N-methylmorpholine, amino acids, and other amines which have been used to form salts of carboxylic acids and phosphoric acids.

Suitable base salts are formed from bases which form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see Handbook of Pharmaceutical Salts: Properties, Selection, and Use by Stahl and Wermuth (Wiley-VCH, 2002). In addition to the methods described herein, methods for making pharmaceutically acceptable salts of phosphates and boronates are known to one of skill in the art.

The compounds of Formula (1) wherein Q is P(O)(OH)(O⁻M⁺), —P(O)(O⁻M⁺)₂ or —P(O)(O⁻)₂M²⁺ can be prepared in a routine manner by admixture of a Formula (1) compound wherein Q is —P(O)(OH)₂ with the appropriate selected base, preferably by contact in solution employing an an excess of commonly used solvent inert solvents such as water, ether, acetonitrile, dioxane, methylene chloride, isopropanol, methanol, ethanol and ethyl acetate. The compounds of Formula (1) wherein Q is P(O)(OH)(O-M⁺), —P(O)(O⁻M⁺)₂ or —P(O)(O⁻)₂M²⁺ can also be prepared by metathesis or by treatment with an ion exchange resin under conditions in which a monovalent cation, M⁺, or divalent cation, M²⁺, in a compound of Formula I is replaced by another monovalent cation, M⁺, or divalent cation, M²⁺, as appropriate, under conditions which allow for separation of the desired species, such as by precipitation from solution or extraction into a solvent, or elution from or retention on an ion exchange resin. Likewise, the compounds of Formula (2) can also be prepared by metathesis or by treatment with an ion exchange resin under conditions in which a monovalent cation, M⁺, in a compound of Formula (2) is replaced by another monovalent cation, M⁺, under conditions which allow for separation of the desired species, such as by precipitation from solution or extraction into a solvent, or elution from or retention on an ion exchange resin.

The compounds of the Formula (1) possess an asymmetric center, thus existing as two stereoisomeric forms. The present invention includes all the individual stereoisomers of the compounds of Formula (1) and mixtures thereof. Individual enantiomers can be obtained by chiral separation or using the relevant enantiomer in the synthesis. For example, the individual (R) and (S) enantiomers of the compound of Formula (1) can be obtained by chiral separation from an enantiomeric mixture or they can be prepared individually using a chiral synthetic method. A preferred embodiment is the compound of Formula Ia in which the compound has the (R) stereochemistry at the chiral carbon center. Similarly, the compounds of Formula (2) also have an asymmetric center and preferred embodiments are the compounds of Formula IIa which has the stereochemistry as depicted.

In addition, the compounds of the present invention can exist in unsolvated as well as solvated forms with pharmaceutically acceptable solvents such as water, ethanol and the like. In general, the solvated forms are considered equivalent to the unsolvated forms for the purposes of the present invention. The compounds may also exist in one or more crystalline states, i.e. polymorphs, or they may exist as amorphous solids. All such forms are encompassed within the scope of the present invention and by the claims.

The compounds of the present invention act as prodrugs of (R)-4-(4-(4-(2H-1,2,3-triazol-2-yl)phenyl)-2-oxopyridin-1(2H)-yl)-N-hydroxy-2-methyl-2-(methylsulfonyl) butanamide; (2R)-4-[4-(2,3-difluoro-4-methoxyphenyl)-2-oxopyridin-1(2H)-yl]-N-hydroxy-2-methyl-2-(methylsulfonyl)butanamide; (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamide; (2R)—N-hydroxy-2-methyl-2-(methylsulfonyl)-4-{2-oxo-4-[4-(1,3-thiazol-2-yl)phenyl]pyridin-1(2H)-yl}butanamide; and (R)-4-(4-(4-(2H-1,2,3-triazol-2-yl)phenyl)-6-oxopyrimidin-1(6H)-yl)-N-hydroxy-2-methyl-2-(methylsulfonyl)butanamide or of the racemates of these compounds. These compounds may have little or no pharmacological activity themselves but when administered into or onto the body, can be converted into the parent compound having the desired activity, for example, by hydrolytic cleavage of the phosphate in compounds of Formula (1) or of the boronate moiety in the compound of Formula (2).

This invention also encompasses compounds containing protective groups. For example, certain intermediate compounds used to prepare compounds of Formula (1) or Formula (2) may contain protecting groups. One skilled in the art will also appreciate that compounds of the present invention can also be prepared with certain protecting groups that are useful for purification or storage and can be removed before administration to a patient. The protection and deprotection of functional groups is described in “Protective Groups in Organic Chemistry”, edited by J. W. F. McOmie, Plenum Press (1973) and “Protective Groups in Organic Synthesis”, 3rd edition, T. W. Greene and P. G. M. Wuts, Wiley-Interscience (1999).

The present invention also includes isotopically-labeled compounds, which are identical to those recited in Formula (1) or Formula (2) but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, fluorine and chlorine, such as, but not limited to, ²H, ³H, ¹³C, ¹⁴C, ¹⁵N, ¹⁷O, ¹⁸O, ³¹P, ³²P, ³⁵S, ¹⁸F, and ³⁶Cl, respectively.

Compounds of the present invention which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds of this invention can generally be prepared by carrying out the procedures disclosed in the Schemes and/or in the Examples below, by substituting a readily available isotopically-labeled reagent for a non-isotopically-labeled reagent.

All of the compounds of Formula (1) contain a sulfonyl moiety as depicted below:

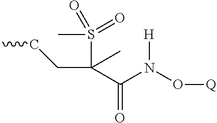

As is readily apparent to one skilled in the art, the carbon adjacent to the sulfonyl moiety is a chiral center. Therefore, the compounds can exist as the racemate, as the S-enantiomer, or as the R-enantiomer or as mixtures thereof. In a further embodiment, the compounds of Formula (1) may be prepared and administered as the R-enantiomer (i.e., a Formula (1a) compound, as depicted below:

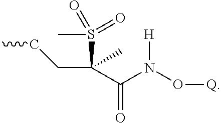

The compounds of Formula (1) and Formula (2) as depicted can be racemic, individual isomers or mixtures thereof whereas the compounds of Formula (1a) and Formula (2a) have the stereochemistry as depicted for those formula, respectively. As is readily apparent to one skilled in the art, the compounds as synthesized will rarely be present exclusively as a single enantiomer. The opposite enantiomer (i.e the S-enantiomer) may be present in minor amounts (i.e. “substantially pure”). This minor amount can be up to 10 w/w %, more typically no greater than 5 w/w %, in a further embodiment no greater than 1 w/w %, or more specifically, no greater than 0.5 w/w %.

### Experimental Synthesis

The compounds of Formula (1) and Formula (2) can be prepared by a variety of methods that are analogously known in the art. The reaction schemes A and B presented below illustrate two alternative methods for preparing the intermediate compounds of Formula I′ or I″. Others, including modifications thereof, will be readily apparent to one skilled in the art. The compounds of Formula I′ or I″ can then be employed in the synthesis of compounds of Formula (1) and Formula (2).

The synthesis of the compounds of Formula I′ or I″ is depicted below in Schemes A and B below. The first step is to carry out the N-alkylation depicted in Step A. The pyridinone/pyrimidinone (where X is CH or N, respectively) of structure 1 is reacted with the sulfonyl derivative of structure 2 generating the intermediate of structure 3. Structure 3 can be further derivatized to generate the compounds of Formula (1). Two alternative syntheses are depicted (Option A or B), but the reader will readily note they are variations of the same synthesis. The only difference is the order in which the steps are carried out.

Initially in Option A, an appropriate leaving group such as a halide, depicted by Lg, at the 4-position of the pyridinone/pyrimidinone of structure 3 is displaced by the desired group Z moiety by reaction with Z-M¹, in which M¹ is a metal species, such as a boron derivative suitable for undergoing a typical cross-coupling such as a Suzuki-Miyaura reaction. Hydrolysis, or removal, of the ethyl protecting group (or other suitable protecting groups) in Step C affords the compound of structure 5. The terminal carboxylic acid of structure 5 is then converted to the protected hydroxamic acid derivative as depicted by structure 8 (wherein Pr is an appropriate protecting group). Deprotection of the protected hydroxamic acid derivative of structure 8, as depicted in Step H, affords the intermediate of Formula I′. While these reactions are well known to one skilled in the art, they are discussed in greater detail below.

Initially, in Option B of Scheme A, the ethyl protecting group (or other conventional protecting groups) is removed from the pyridinone/pyrimidinone of structure 3 generating the compound of structure 6 as depicted in Step E. In Step F, the terminal carboxylic acid of structure 6 is converted to the protected hydroxamic acid derivative of structure 7 via amidation conditions. In Step G, the leaving group Lg such as a halide function on the pyridinone/pyrimidinone moiety is then directly displaced by the desired group Z moiety, by reacting Z-M¹, via a coupling reaction to afford the protected hydroxamic acid derivatives of structure 8. As before, deprotection of the protected hydroxamic acid derivatives, as depicted in Step H, affords the compounds of Formula I′.

Scheme B, depicted below, is analogous to Scheme A with the exception that the pyridinone/pyrimidinone of structure 1 is reacted with the sulfonyl derivative of structure 2′ generating the intermediate of structure 3′. Structure 3′ can be further derivatized to generate the compound of Formula I″. Initially in Option A, an appropriate leaving group such as halide, depicted by Lg, on the 2-pyridinone/pyrimidinone of structure 3′ is displaced by the desired Z moiety by reaction with Z-M¹, in which M¹ is a metal species, such as a boron derivative suitable for undergoing a typical cross-coupling such as a Suzuki-Miyaura reaction. Hydrolysis, or removal, of the ethyl protecting group (or other suitable protecting groups) in Step C affords the compound of structure 5′. The terminal carboxylic acid of structure 5′ is then converted to the protected hydroxamic acid derivative as depicted by structure 8′ (wherein Pr is an appropriate protecting group). Deprotection of the protected hydroxamic acid derivative of structure 8′, as depicted in Step H, affords the intermediate of Formula I″. While these reactions are well known to one skilled in the art, they are discussed in greater detail below.

Initially, in Option B of Scheme B, the ethyl protecting group (or other conventional protecting groups) is removed from the pyridinone/pyrimidinone of structure 3′ generating the compound of structure 6′ as depicted in Step E. In Step F, the terminal carboxylic acid of structure 6′ is converted to the protected hydroxamic acid derivative of structure 7′ via amidation conditions. In Step G, an appropriate leaving group Lg, such as a halide function on the pyridinone/pyrimidinone moiety is then directly displaced by the desired group Z moiety, by reacting Z-M¹, via a coupling reaction to afford the protected hydroxamic acid derivatives of structure 8′. As before, deprotection of the protected hydroxamic acid derivatives, as depicted in Step H, affords the compounds of Formula I″.

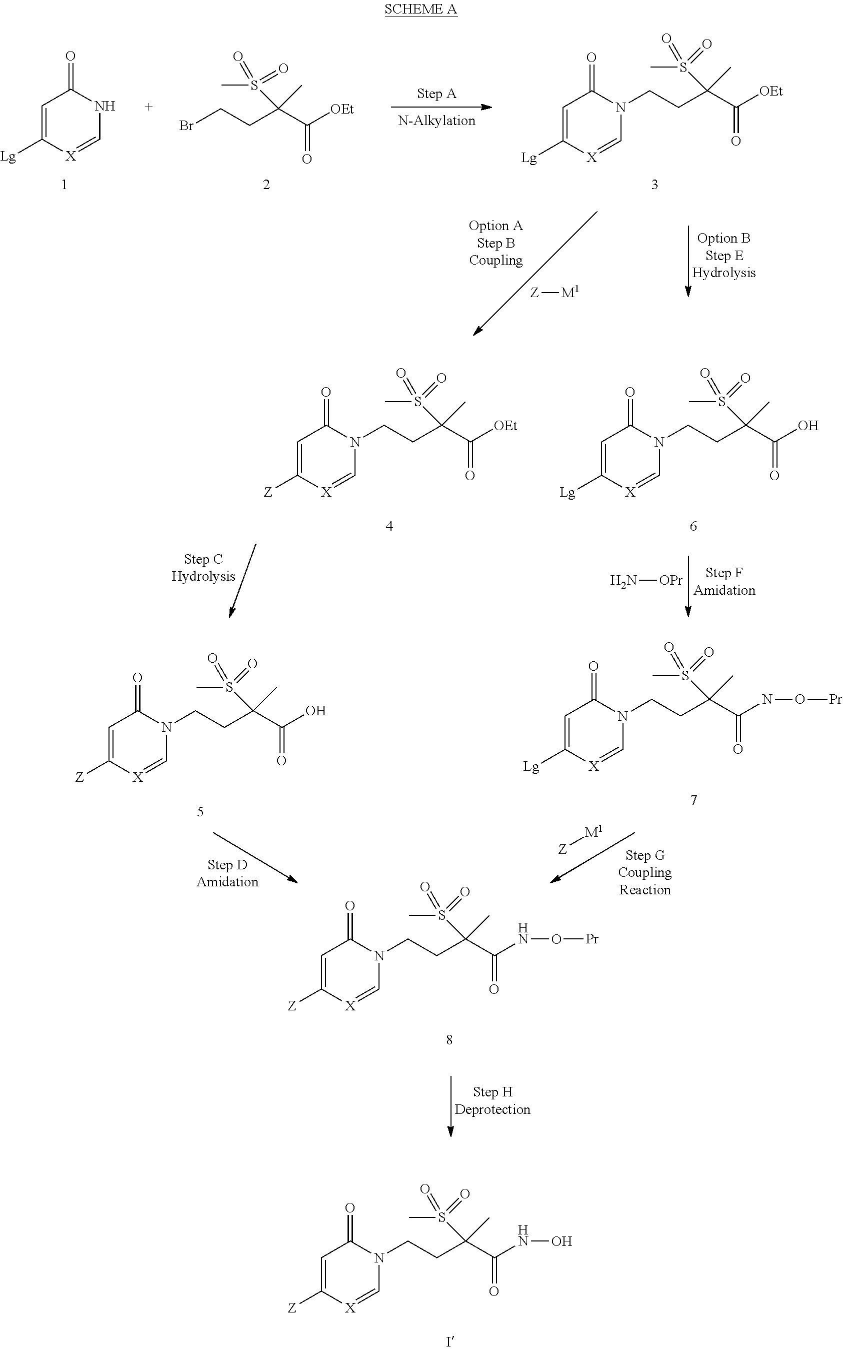

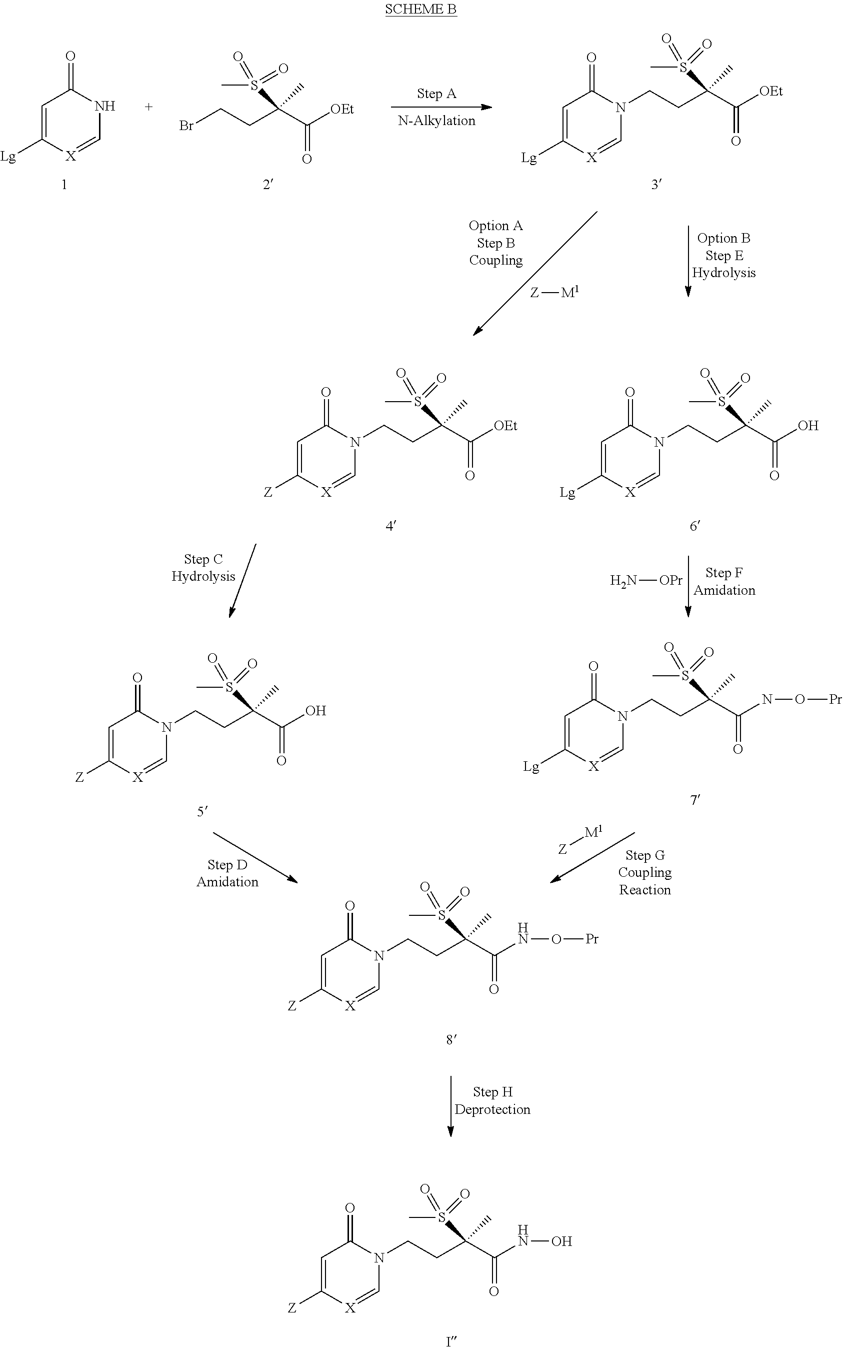

The following description relates to the synthetic steps used in Schemes A and B. The N-alkylation depicted above in Step A of Scheme A and Scheme B can be carried out using techniques well known to one skilled in the art. One of the starting materials is the 2-pyridinone or pyrimidinone derivative of structure 1. In this pyridinone or pyrimidinone, Lg is an appropriate leaving group such as a halide. Many of these pyridinone or pyrimidinone derivatives are known in the art and the remainder can be produced using synthetic techniques analogously known in the art. The reader's attention is directed to Tet. Lett. (2005) Vol 46, 7917, for a description of such techniques. Preparation 2 infra, also illustrates their preparation.

The other reactant in the N-alkylation depicted in Step A is the protected alkyl sulfonate of structure 2 or 2′. In structure 2 or 2′ an ethyl protecting group is portrayed (i.e. protecting the carboxylic acid as its ethyl ester), but any standard carboxylic acid protecting group may be substituted. These alkyl sulfonates are also known in the art. The reader's attention is directed to Journal of Organic Chemistry, (1980) Vol 45, 8, 1486-1489 for a description of their preparation. Preparation 1 infra, also illustrates their preparation.

The N-alkylation can be carried out as is known in the art. Typically, equivalent amounts of the compounds of structure 1 and 2 or 2′ are contacted in a mixture of aprotic and protic solvents, such as tetrahydrofuran and t-butanol, in the presence of a base such as potassium carbonate, cesium carbonate, sodium carbonate, sodium hydride, etc. A transfer agent, such as tetrabutyl ammonium bromide, can be utilized, if desired. The reactants are typically heated and the reaction is allowed to proceed to completion. The desired product of structure 3 or 3′ can be isolated by methods known in the art. If desired, the product of structure 3 or 3′ can be purified, or alternatively the crude can be used in the next step of the reaction. Preparation 2 infra, illustrates such an N-alkylation.

Scheme A illustrates how to incorporate the hydroxamic acid moiety into the molecules. Initially, the protecting group is removed from the carboxylic acid, thereby generating the intermediate of structure 5 or 5′ and 6 or 6′, as depicted in Step C (Option A) and Step E (Option B) respectively. The manner in which this is accomplished will vary with the identity of the actual protecting group and is well known to those skilled in the art. The reader's attention is directed to McOmie or Greene supra, for a discussion of potential protecting groups and methods for their removal. Preparation 2 infra describes how to remove an ethyl moiety as depicted in Schemes A and B.

In Steps F and D, the hydroxamic acid moiety as depicted, is incorporated into the molecule. A protected hydroxylamine source may be used followed by a subsequent deprotection reaction (alternatively, hydroxylamine may be directly incorporated to eliminate the deprotection steps). In either case the hydroxamic acid is incorporated into the molecule using standard amidation reactions. For example, the compound of structure 5 or 5′ (Option A) or 6 or 6′ (Option B) may be contacted with an excess of oxalyl chloride, in an aprotic solvent such as dichloromethane for a sufficient period of time to allow the formation of the corresponding acid chloride, followed by the addition of an excess of either hydroxylamine or protected hydroxylamine. The reaction is then allowed to proceed to completion and the protected intermediates of structure 7 or 7′ (Option B) or 8 or 8′ (Option A) is isolated from the reaction medium and purified as is known in the art. As mentioned above, any deprotection may be carried out as is known in the art (See Greene or McOmie supra). Alternatively, the amide can be formed using the amide coupling reagent, 1,1′-carbonyldiimidazole (CDI), 2-chloro-4,6-dimethoxy-1,3,5-triazine (CDMT), or 1-ethyl-3-(3-dimethylaminopropyl)carbodiimide (EDCI), as is known in the art.

Schemes A and B also depict how to incorporate the terminal group Z moiety, into the molecule. Regardless of whether Option A or Option B is chosen, a coupling reaction is ultimately carried out to attach the terminal group Z moiety, to the pyridinone/pyrimidinone intermediate. In both Scheme A and B, the co-reactant is depicted as Z-M¹, where M¹ represents a metal (or metalloid) such as magnesium, copper, tin, boronic ester/acid, etc. at the desired point of attachment to the pyridinone/pyrimidinone intermediate of structure 3 or 3′ or 7 or 7′ (i.e. the other reactant).

The coupling reaction can be carried out by a variety of techniques. The Suzuki-Miyaura strategy can be used to form the carbon-carbon bond. In such a reaction M¹ will be represented by a boronic acid/ester. Equivalent molar amounts of the reactants will be contacted in a solvent such as tetrahydrofuran, 2-methyltetrahydrofuran, 1,4-dioxane, water, toluene, or a mixture thereof in the presence of a transition metal catalyst such as a free or resin bound palladium or nickel species, together with a base such as sodium carbonate, potassium carbonate, cesium fluoride, cesium carbonate, etc. The reaction mixture can be heated by microwave or by other conventional techniques until adequate conversion is achieved. Once complete, the desired product may be isolated and recovered from the reaction and further purified as is known in the art. Analogously, other carbon-carbon bond forming methods known in the art can be employed to carry out the coupling reaction. In such a reaction M¹ can be represented by an in situ generated cuprate species or a trialkyl tin moiety, such as trimethylstannyl, tributylstannyl or tri-t-butylstannyl. Equivalent molar amounts of the reactants will be contacted in a solvent such as tetrahydrofuran, 2-methyltetrahydrofuran, dimethylformamide or a mixture thereof in the presence of a transition metal catalyst such as free or resin bound palladium or nickel, together with an appropriate base such as a suitable organic base for example N,N-diisopropylethylamine. The reaction mixture can be heated by microwave or by other conventional techniques until adequate conversion is achieved. Once complete, the desired product may be isolated and recovered from the reaction and further purified as is known in the art.

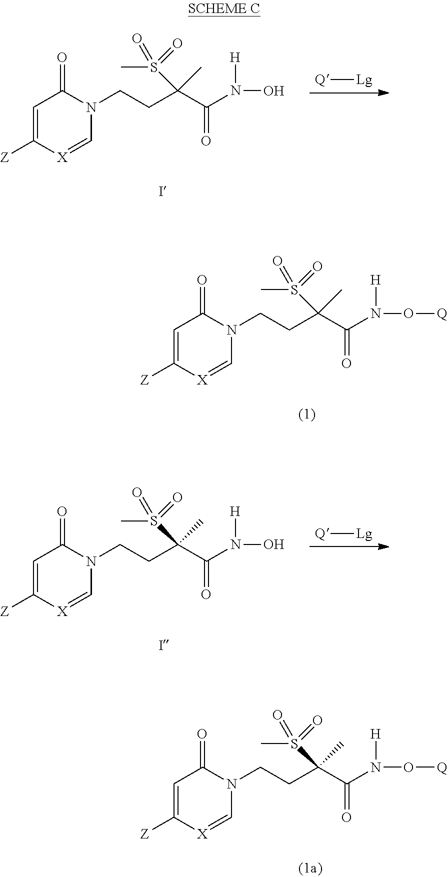

Scheme C depicts the preparation of compounds of Formula (1) and Formula (1a) from compounds I′ and I″, respectively. The compound of Formula I′ or I″ is reacted with an appropriate phosphate precursor compound, Q′-Lg, wherein Lg represents an appropriate leaving group and Q′ represents a phosphorous containing group that can be converted to an appropriate phosphate group Q. Examples of phosphate precursor compounds Q′-Lg include phosphorous oxychloride (POC) or a phosphoramidite reagent (PgO)₂P—NR′₂. Under appropriate reaction conditions the Q′ moiety is converted into the group Q as set forth in Formula (1) or Formula (1a). A more detailed description of such conversions of Q′ to Q is provided below in Schemes D and E.

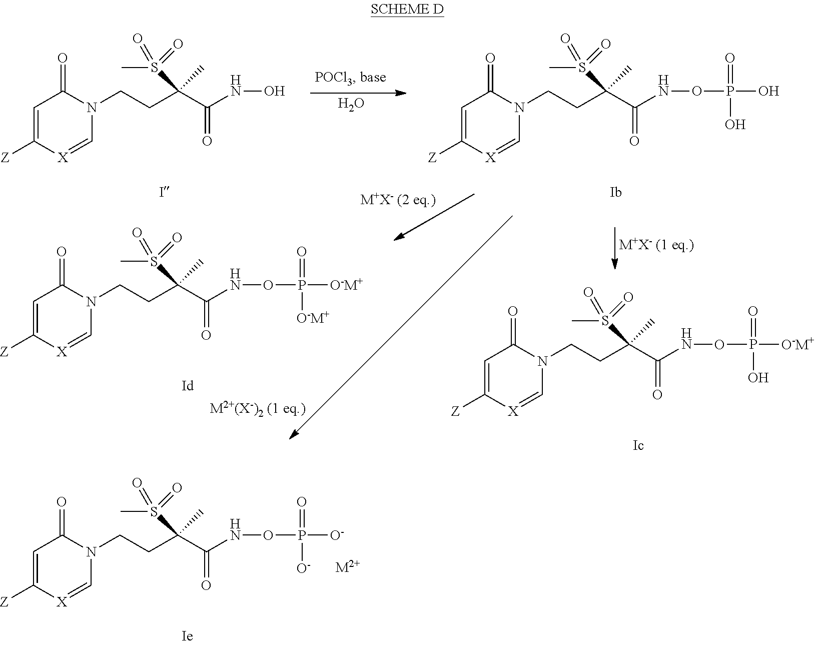

Scheme D depicts the preparation of novel phosphates within the scope of Formula (1) (i.e. compounds of Formula Ib, Ic, Id and Ie). The hydroxamic acid compound of Formula I″ is dissolved in an appropriate solvent, such as acetonitrile, and treated with an appropriate base, such as N-methylmorpholine at a reduced temperature, such as 0° C. to −10° C. The resulting mixture is then reacted with phosphorous oxychloride and can then be quenched with water to provide the phosphate of Formula Ib. The compound of Formula Ib can then be reacted with an appropriate base (i.e. M⁺X⁻ or M²⁺(X⁻)₂ wherein X⁻ is an anionic counterion) as shown to provide the compounds of Formula Ic, Id or Ie. Alternatively, the compound of formula Ib could be treated with an appropriate ion exchange resin, such as a Dowex ion exchange resin, in an aqueous solution to provide a compound of formula Id.

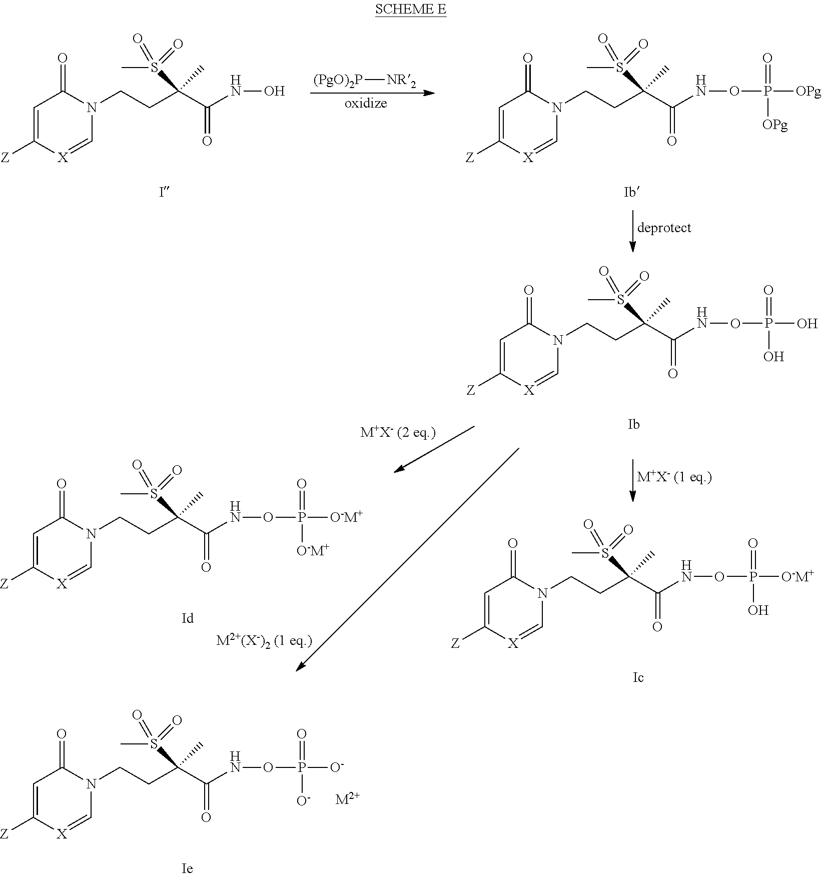

Scheme E depicts an alternative method for preparing the compounds of Formula Ib-Ie. The compound of Formula I″ is reacted with a suitable phosphoramidite reagent, (PgO)₂P—NR′₂, in which the group Pg represents an appropriate protecting group such as t-butyl or benzyl and the group R′ represents a lower alkyl group such as ethyl or isopropyl. The reaction is typically carried out at approximately ambient temperature in an appropriate solvent such as acetonitrile, dichloromethane or a mixture thereof in the presence of an activating agent such as tetrazole for a period of one to eight hours. The reaction mixture can then be cooled and in situ oxidation carried out by treatment with an appropriate oxidizing agent such as hydrogen peroxide, t-butyl hydroperoxide or m-CPBA to provide the compound of Formula Ib′. The compound of Formula Ib′ is then deprotected using standard methodology to provide the compounds of Formula Ib. For example, when Pg represents t-butyl the compound of Formula Ib′ can be deprotected by treatment with a strong acid such as hydrochloric acid or trifluoroacetic acid. Alternatively, when Pg represents benzyl the compound of Formula Ib′ can be deprotected by catalytic hydrogenation. The compound of Formula Ib can then be used to prepare the compounds of Formula Ic, Id or Ie as previously described for Reaction Scheme D.

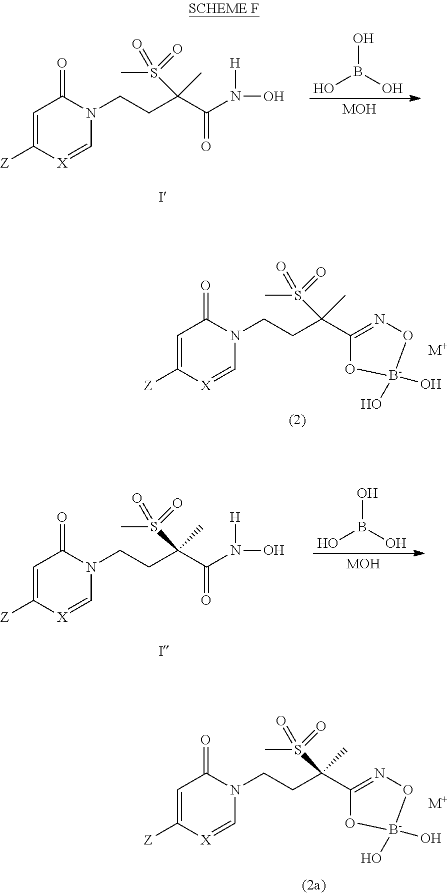

Scheme F depicts the preparation of the borate monomer compounds of Formula (2) and Formula (2a). One equivalent of the hydroxamic acid of Formula I′ or I″ is combined with one equivalent of boric acid in water in the presence of one equivalent of an appropriate base such as sodium hydroxide, potassium hydroxide or lithium hydroxide (MOH). The mixture is stirred at ambient temperature for 30 minutes to four hours then the mixture can be either concentrated in vacuo or frozen and lyophilized to provide the monoboronate compound of Formula (2) or Formula (2a).

The reaction schemes depicted above for producing the compounds of the present invention are merely illustrative. As is readily apparent to one skilled in the art, they may be modified depending upon the specific compound, availability of reagents, etc.

### Medical and Veterinary Uses

The compounds of the present invention may be used for the treatment or prevention of infectious disorders, especially those caused by susceptible and multi-drug resistant (MDR) Gram-negative bacteria. Examples of such Gram-negative bacteria include Acinetobacter baumannii, Acinetobacter spp., Achromobacter spp., Aeromonas spp., Bacteroides fragilis, Bordetella spp., Borrelia spp., Brucella spp., Campylobacter spp., Citrobacter diversus (koseri), Citrobacter freundii, Enterobacter aerogenes, Enterobacter cloacae, Escherichia coli, Francisella tularensis, Fusobacterium spp., Haemophilus influenzae (β-lactamase positive and negative), Helicobacter pylori, Klebsiella oxytoca, Klebsiella pneumoniae (including those encoding extended-spectrum β-lactamases (hereinafter “ESBLs”), Legionella pneumophila, Moraxella catarrhalis (β-lactamase positive and negative), Morganella morganii, Neisseria gonorrhoeae, Neisseria meningitidis, Proteus vulgaris, Porphyromonas spp., Prevotella spp., Mannheimia haemolyticus, Pasteurella spp., Proteus mirabilis, Providencia spp., Pseudomonas aeruginosa, Pseudomonas spp., Salmonella spp., Shigella spp., Serratia marcescens, Treponema spp., Burkholderia cepacia, Vibrio spp., Yersinia spp., and Stenotrophomonas mulophilia. Examples of other gram negative organisms include members of the Enterobacteriaceae that express ESBLs; KPCs, CTX-M, metallo-β-lactamases (such as NDM-1, for example), and AmpC-type beta-lactamases that confer resistance to currently available cephalosporins, cephamycins, carbapenems, and beta-lactam/beta-lactamase inhibitor combinations.

In a more specific embodiment, the Gram-negative bacteria are selected from the group consisting of Acinetobacter baumannii, Acinetobacter spp., Citrobacter spp., Enterobacter aerogenes, Enterobacter cloacae, Escherichia coli, Klebsiella oxytoca, Klebsiella pneumoniae, Serratia marcescens, Stenotrophomonas maltophilia, Pseudomonas aeruginosa and members of the Enterobacteriaceae and Pseudomonas that express ESBLs, KPCs, CTX-M, metallo-β-lactamases, and AmpC-type beta-lactamases that confer resistance to currently available cephalosporins, cephamycins, carbapenems, and beta-lactam/beta-lactamase inhibitor combinations.

Examples of infections that may be treated with the compounds of Formula (1) include nosocomial pneumonia, urinary tract infections, systemic infections (bacteremia and sepsis), skin and soft tissue infections, surgical infections, intraabdominal infections, lung infections in patients with cystic fibrosis, patients suffering from lung infections, endocarditis, diabetic foot infections, osteomyelitis, and central nervous system infections.

In addition, the compounds can be used to treat Helicobacter pylori infections in the GI tract of humans (and other mammals). Elimination of these bacteria is associated with improved health outcomes including fewer dyspeptic symptoms, reduced peptic ulcer recurrence and rebleeding, reduced risk of gastric cancer, etc. A more detailed discussion of eradicating H. pylori and its impact on gastrointestinal illness may be found on the world wide web at: informahealthcare.com, Expert Opin. Drug Saf. (2008) 7(3).

In order to exhibit this anti-infective activity, the compounds need to be administered in a therapeutically effective amount. A “therapeutically effective amount” is meant to describe a sufficient quantity of the compound to treat the infection, at a reasonable benefit/risk ratio applicable to any such medical treatment. It will be understood, however, that the attending physician, within the scope of sound medical judgment, will decide the total daily dosage of the compound. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. As a general guideline however, the total daily dose will typically range from about 0.1 mg/kg/day to about 5000 mg/kg/day in single or in divided doses. Typically, dosages for humans will range from about 10 mg to about 3000 mg per day, in a single or multiple doses.

Any route typically used to treat infectious illnesses, including oral, parenteral, topical, rectal, transmucosal, and intestinal, can be used to administer the compounds. Parenteral administrations include injections to generate a systemic effect or injections directly into to the afflicted area. Examples of parenteral administrations are subcutaneous, intravenous, intramuscular, intradermal, intrathecal, and intraocular, intranasal, intravetricular injections or infusions techniques. Topical administrations include the treatment of areas readily accessible by local application, such as, for example, eyes, ears including external and middle ear infections, vaginal, open wound, skin including the surface skin and the underneath dermal structures, or lower intestinal tract. Transmucosal administration includes nasal aerosol or inhalation applications. Oral administration includes, tablets, capsules, solutions, suspensions, admixture with water and/or food, saches, and the like.

### Formulations

Compounds of the present invention can be formulated for administration in any way for use in human or veterinary medicine, by analogy with other bioactive agents such as antibiotics. Such methods are known in the art and are summarized below.

The composition can be formulated for administration by any route known in the art, such as subdermal, by—inhalation, oral, topical or parenteral. The compositions may be in any form known in the art, including but not limited to tablets, capsules, powders, granules, lozenges, creams or liquid preparations, such as oral or sterile parenteral solutions or suspensions.

The topical formulations of the present invention can be presented as, for instance, ointments, creams or lotions, ophthalmic ointments/drops and otic drops, impregnated dressings and aerosols, and may contain appropriate conventional additives such as preservatives, solvents to assist drug penetration and emollients, etc. Such topical formulations may also contain conventional carriers, such as cream or ointment bases and ethanol or oleyl alcohol for lotions. Such carriers may be present, for example, from about 1% up to about 98% of the formulation.

Tablets and capsules for oral administration may be in unit dose presentation form, and may contain conventional excipients such as binding agents, for example acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being typical. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% by weight, to about 100% by weight, of the active material, depending on the method of administration. Where the compositions comprise dosage units, each unit will contain, for example, from about 0.5-1000 mg of the active ingredient. The dosage as employed for adult human treatment will range, for example, from about 10 to 3000 mg per day, depending on the route and frequency of administration.

If desired, the compounds of the present invention may be administered in combination with one or more additional antibacterial agents (“the additional active agent”). Such use of compounds of the present invention in combination with an additional active agent may be for simultaneous, separate or sequential use.

The Examples and preparations provided below further illustrate and exemplify the compounds of the present invention and methods of preparing such compounds. It is to be understood that the scope of the present invention is not limited in any way by the scope of the following Examples and preparations. In the following Examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### EXAMPLES

### Experimental Procedures

Experiments were generally carried out under an inert atmosphere (nitrogen or argon), particularly in cases where oxygen- or moisture-sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Mass spectrometry data is reported from either liquid chromatography-mass spectrometry (LCMS) or atmospheric pressure chemical ionization (APCI). Chemical shifts for nuclear magnetic resonance (NMR) data are expressed in parts per million (ppm, b) referenced to residual peaks from the deuterated solvents employed. Melting points are uncorrected. Low Resolution Mass Spectra (LRMS) were recorded on either a Hewlett Packard 5989®, utilizing chemical ionization (ammonium), or a Fisons (or Micro Mass) Atmospheric Pressure Chemical Ionization (APCI) platform which uses a 50/50 mixture of acetonitrile/water with 0.1% formic acid as the ionizing agent. Room or ambient temperature refers to 20-25° C.

For syntheses referencing procedures in other Examples, reaction conditions (length of reaction and temperature) may vary. In general, reactions were followed by thin layer chromatography or mass spectrometry, and subjected to work-up when appropriate. Purifications may vary between experiments: in general, solvents and the solvent ratios used for eluents/gradients were chosen to provide appropriate R_{f}s or retention times.

In the discussion above and in the Examples below, the following abbreviations have the following meanings. If an abbreviation is not defined, it has its generally accepted meaning: atmospheric pressure chemical ionization (APCI); aqueous (aq); deuteron chloroform (CDCl₃); 2-chloro-4,6-dimethoxy-1,3,5-triazine (CDMT); deuteron methanol (CD₃OD); dichloromethane (DCM); dimethylformamide (DMF); dimethyl sulfoxide (DMSO); ethyl acetate (EtOAc); grams (g); hours (h, hr, hrs); hydrochloric acid (HCl); high pressure liquid chromatography (HPLC); potassium hydroxide (KOH); liquid chromatography mass spectrometry (LCMS); leaving group (Lg); lithium hydroxide (LiOH); meta-chloroperbenzoic acid (mCPBA); magnesium sulfate (MgSO₄); minutes (min); sodium hydroxide (NaOH); palladium (Pd); palladium acetate and BINAP, microencapsulated in polyurea matrix 0.39 mmol/g Pd loading BINAP 0.25, Pd 1.0 (Pd EnCat™); bis(diphenylphosphino)ferrocenepalladium(II) chloride (Pd(dppf)Cl₂); retention factor (R_{f}); retention time (rt); room temperature (RT); trifluoroacetic acid (TFA); tetrahydrofuran (THF); tetrahyropyranyl (THP); tetramethylsilane (TMS); theoretical yield (TY); and uridine 5′-diphosphate (UDP).

### Preparation of Starting Materials

### Preparation 1 and Preparation 1A

### (+/−)-Ethyl 4-bromo-2-methyl-2-(methylsulfonyl)butanoate and Individual Enantiomers (R) and (S)

### Step A) Ethyl 2-(methylsulfonyl)propanoate

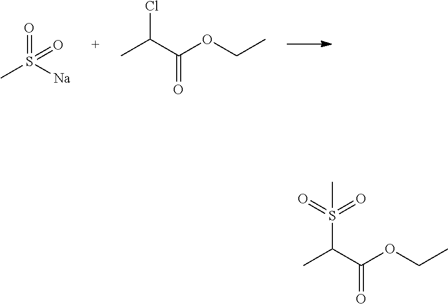

Sodium methyl sulfinate (103 g, 937 mmol) was combined with the ethyl 2-chloropropionate (109 g, 892 mmol) in ethanol (350 mL) in a 500 mL one neck round bottom flask. The reaction was warmed to 77° C. for 20 hours, and then allowed to cool to room temperature. Solids were removed by filtration through celite, and the filter pad was washed with ethanol and the combined filtrates were concentrated in vacuo. The crude product was suspended in diethyl ether (250 mL), and solids were removed by filtration. The filtrate was concentrated in vacuo to afford the title compound as a pale yellow oil (51 g, 73%). ¹H NMR (CDCl₃, 400 MHz) δ ppm 1.32 (t, J=7.05 Hz, 3H) 1.67 (d, J=7.47 Hz, 3H) 3.05 (s, 3H) 3.83-3.92 (m, 1H) 4.18-4.37 (m, 2H).

### Step B) (+/−)-Ethyl 4-bromo-2-methyl-2-(methylsulfonyl)butanoate

Sodium hydride (60% dispersion in mineral oil, 2.33 g, 58.3 mmol) was washed with hexane (2×10 mL) in a 100 mL two neck round bottom flask under nitrogen then suspended in DMF (30 mL). The suspension was treated dropwise with ethyl 2-(methylsulfonyl)propanoate (10.0 g, 55.49 mmol) in DMF (10 mL). The mixture was stirred 30 min at RT, cooled to 0° C., and treated dropwise with 1,2-dibromoethane (5.17 mL, 58.8). The mixture was allowed to warm to room temperature while stirring overnight. The mixture was quenched with saturated ammonium chloride (100 mL) and the mixture was extracted with diethyl ether (4×50 mL). Combined organics were washed with 50% saturated sodium chloride (4×50 mL), dried (MgSO₄), filtered and the filtrate concentrated in vacuo. Crude material was chromatographed over silica gel (350 g, 230-400 mesh) eluting with 10-20% EtOAc/hexane to afford the title compound as a pale yellow oil (7.9 g, 50%). ¹H NMR (CDCl₃, 400 MHz) δ ppm 1.33 (t, J=7.05 Hz, 3H) 1.64 (s, 3H) 2.49-2.59 (m, 1H) 2.78 (ddd, J=13.89, 10.16, 6.64 Hz, 1H) 3.05 (s, 3H) 3.33-3.41 (m, 1H) 3.46-3.54 (m, 1H) 4.22-4.37 (m, 2H).

### Step C) Chiral Separation of (+/−)-Ethyl 4-bromo-2-methyl-2-(methylsulfonyl)butanoate

Crude (+/−)-ethyl 4-bromo-2-methyl-2-(methylsulfonyl)butanoate (1.82 kg) was purified via flash chromatography using an LP-600 column and toluene as the eluant to afford pure (+/−)-ethyl 4-bromo-2-methyl-2-(methylsulfonyl)butanoate (1.63 kg). The purified material was dissolved in ethanol (75 g/L) and resolved via chiral multi-column chromatography (condition listed in Table 1) on MCC-2 to afford enantiomer 1 (738.4 g, rt=4.719 min, [α]₅₈₉²⁰=+14.1°) at 99% enantiomeric purity and enantiomer #2 (763.8 g, rt=4.040 min) at 95% enantiomeric purity. Purity of the enantiomers was determined via chiral HPLC, 4.6×250 mm Chiralpak AD, 10μ column, 215 nm wavelength, mobile phase: ethanol, isocratic elution at 1 mL/min at ambient temperature.

TABLE 1

| Stationary Phase | ChiralPak AD, 20 μ | |
|---|---|---|
| Column Dimension/ | 5 × 10 cm/ | |
| Temp | 30° C. | |
| Mobile Phase | 100% ethanol | |
| Feed Concentration | 75 g/L in | |
| | mobile phase | |
| Feed Rate | 4.0 | mL/min |
| Eluant Rate | 90.5 | mL/min |
| Raffinate Rate | 35.6 | mL/min |
| Extract Rate | 58.9 | mL/min |
| Recycling Rate | 262 | mL/min |
| Period Time | 1.0 | min |

Enantiomer 1 was determined to be Ethyl (2R)-4-bromo-2-methyl-2-(methylsulfonyl)butanoate.

### Preparation 1B

### Benzyl (+/−)-4-bromo-2-methyl-2-(methylsulfonyl)butanoate and Individual enantiomers (R) and (S)

### Step A) Benzyl 2-chloropropanoate

Benzyl alcohol (242 mL, 253 g, 2.34 mol) and pyridine (204 mL, 204 g, 2.57 mol) were dissolved in methylene chloride (2.5 L) and cooled to 0° C. 2-Chloropropanoyl chloride (250 mL, 327 g, 2.57 mol) was added dropwise keeping the temperature between 0° C. and 5° C. After addition the mixture was allowed to warm to RT overnight. The mixture was washed with 20% aqueous citric acid (2.5 L), saturated aqueous NaHCO₃ (2.5 L), brine (2.5 L), dried (MgSO₄), filtered and concentrated in vacuo. The resulting brown liquid (450 g) was dissolved in a small amount of methylene chloride and filtered through a short path of silica gel. After concentration, the crude was purified via bulb-to-bulb distillation (2*10-2 mbar, 90-95° C.) affording the title compound as a pale yellow liquid (420 g, 90%). ¹H NMR (CDCl₃, 300 MHz) δ ppm 1.75 (d, 3H, CH₃), 4.45 (q, 1H, CHCl), 5.25 (s, 2H, CH₂Ar), 7.40 (m, 5H, ArH).

### Step B) Benzyl 2-(methylsulfonyl)propanoate

Benzyl 2-chloropropanoate was converted to the title compound following the general procedure outlined for ethyl 2-(methylsulfonyl)propanoate in Preparation 1A. The title compound was obtained as a yellow liquid (389 g, 70%). ¹H-NMR (CDCl₃, 300 MHz) δ ppm 1.65 (dt, 3H, CHCH₃), 3.00 (s, 3H, SO₂CH₃), 3.95 (q, 1H, CH), 5.25 (m, 2H, CO₂CH₂Ar), 7.40 (m, 5H, ArH).

### Step C) Benzyl (+/−)-4-bromo-2-methyl-2-(methylsulfonyl)butanoate

Benzyl 2-(methylsulfonyl)propanoate was converted to the title compound following the general procedure outlined for ethyl (+/−)-4-bromo-2-methyl-2-(methylsulfonyl)butanoate in Preparation 1A. The title compound was obtained as a pale yellow liquid (300 g, 58%). ¹H NMR (CDCl₃, 300 MHz) δ ppm 1.70 (s, 3H, CH₃), 2.60 (m, 1H, CH₂CH₂Br), 2.80 (m, 1H, CH₂CH₂Br), 3.00 (s, 3H, SO₂CH₃), 3.35 (m, 1H, CH₂CH₂Br), 3.50 (m, 1H, CH₂CH₂Br), 5.30 (m, 2H, CO₂CH₂Ar), 7.40 (m, 5H, ArH).

### Step D) Chiral separation of Benzyl (+/−)-4-bromo-2-methyl-2-(methylsulfonyl)butanoate

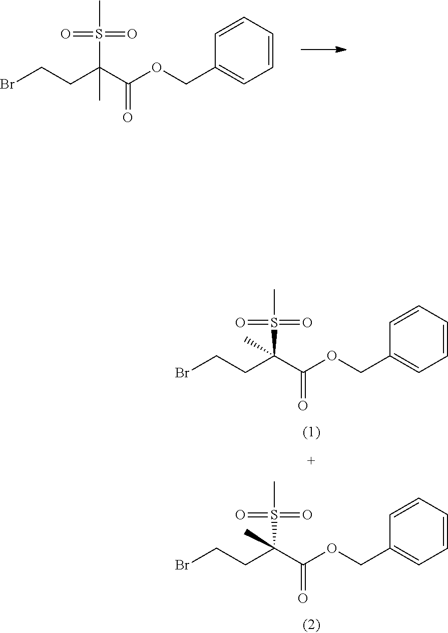 [SMILES: CC(CCBr)(C(=O)OCc1ccccc1)S(C)(=O)=O ; C[C@@](CCBr)(C(=O)OCc1ccccc1)S(C)(=O)=O ; C[C@](CCBr)(C(=O)OCc1ccccc1)S(C)(=O)=O]

Benzyl (+/−)-4-bromo-2-methyl-2-(methylsulfonyl)butanoate (275 g) was dissolved in isopropanol/acetonitrile (900 mL) and resolved using an Analytical SFC-4 instrument, AS-H column (30×250), a CO₂/Propanol (90/10) mobile phase, with a flow rate of 120 g/min to afford enantiomer 1 (98 g, rt=3.09 min, [α]₅₈₉²⁰=−13.9°) at 99.94% enantiomeric purity and enantiomer 2 (101.5 g, retention time=4.18 min, [α]₅₈₉²⁰=+11.61°) at 97.77% enantiomeric purity.

### (S)-benzyl 4-bromo-2-methyl-2-(methylsulfonyl)butanoate

¹H NMR (CDCl₃, 400 MHz) δ ppm 1.65 (s, 3H) 2.48-2.60 (m, 1H) 2.74-2.86 (m, 1H) 2.95 (s, 3H) 3.25-3.37 (m, 1H) 3.40-3.52 (m, 1H) 5.16-5.31 (m, 2H) 7.31-7.40 (m, 5H). [α]₅₈₉²⁰=−13.9°.

### (R)-benzyl 4-bromo-2-methyl-2-(methylsulfonyl)butanoate

¹H NMR (CDCl₃, 400 MHz) δ ppm 1.67 (s, 3H) 2.51-2.61 (m, 1H) 2.75-2.87 (m, 1H) 2.97 (s, 3H) 3.28-3.37 (m, 1H) 3.40-3.60 (m, 1H) 5.15-5.36 (m, 2H) 7.30-7.48 (m, 5H). [α]₅₈₉²⁰=+11.610.

### Preparation 2

The reaction scheme below illustrates the preparation of 4-(4-iodo-2-oxopyridin-1(2H)-yl)-2-methyl-2-(methylsulfonyl)-N-(tetrahydro-2H-pyran-2-yloxy)butanamide and its corresponding R-enantiomer. The reaction sequence in Preparation 2B, is the same with the exception that benzyl (2R)-4-bromo-2-methyl-2-(methylsulfonyl)butanoate is used as a starting material in order to arrive at the desired enantiomer.

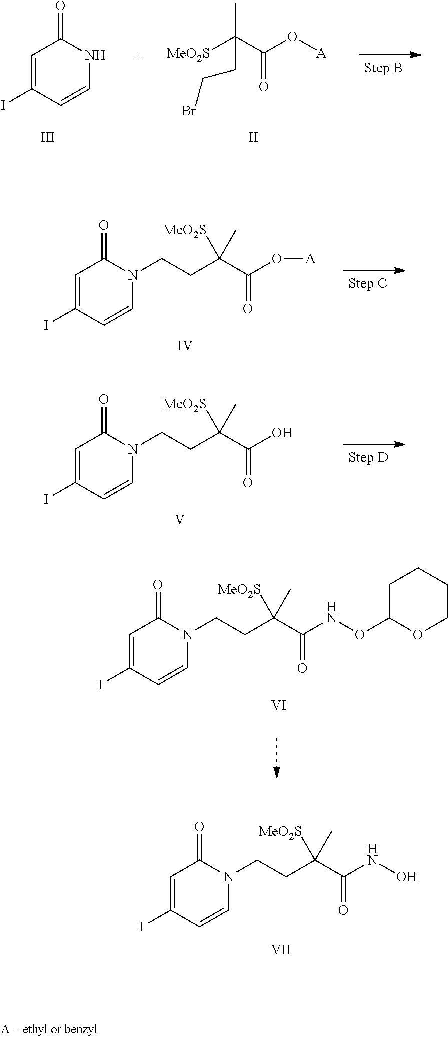

### Synthesis of Compound VI (T3): 4-(4-iodo-2-oxopyridin-1(2H)-yl)-2-methyl-2-(methylsulfonyl)-N-(tetrahydro-2H-pyran-2-yloxy)butanamide as a Mixture of Diastereoisomers

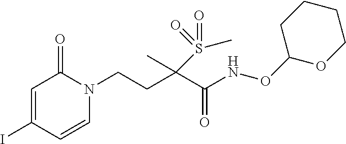 [SMILES: CC(CCn1ccc(I)cc1=O)(C(=O)NOC1CCCCO1)S(C)(=O)=O]

### Step A) 4-iodopyridin-2(1H)-one (Compound III)

2-fluoro-4-iodopyridine (2.21 kg, 9.91 mol) was suspended in a mixture of acetic acid (7 L) and H₂O (3.5 L) with mechanical stirring. The mixture was heated at reflux overnight. After cooling to room temperature the solid was filtered off and concentrated in vacuo. The residue was stirred in Et₂O (3 L), the title compound (1.72 kg, 7.78 mol) was collected by filtration as a pale yellow solid. ¹H NMR (DMSO-d₆, 300 MHz) δ ppm 6.50 (d, 1H), 6.85 (s, 1H), 7.15 (d, 1H), 11.80 (s, 1H).

### Step B) Compound IV(T1): Ethyl 4-(4-iodo-2-oxopyridin-1(2H)-yl)-2-methyl-2-(methylsulfonyl)butanoate (A=Et)

To a mixture of 4-iodopyridin-2(1H)-one (3.9 g, 18 mmol), which may be produced in Step A above, and cesium carbonate (11.9 g, 35.3 mmol) in tetrahydrofuran (176 mL) at ambient temperature was added ethyl 4-bromo-2-methyl-2-(methylsulfonyl)butanoate (6.08 g, 21.2 mmol)(Compound II). The mixture was heated to 50° C. and stirred overnight. The mixture was allowed to cool to ambient temperature and filtered through a celite pad. The pad was washed with methylene chloride and the filtrate was concentrated in vacuo. The crude oil was purified via silica gel chromatography, eluting with heptanes/ethyl acetate. The desired fractions were isolated, the solvent removed via rotary evaporation ethyl 4-(4-iodo-2-oxopyridin-1(2H)-yl)-2-methyl-2-(methylsulfonyl)butanoate as a solid. 4.73 g. LCMS: (M+1) 428.2

### Step C) Compound (V)T2: 4-(4-iodo-2-oxopyridin-1(2H)-yl)-2-methyl-2-(methylsulfonyl)butanoic Acid

To a solution of ethyl 4-(4-iodo-2-oxopyridin-1(2H)-yl)-2-methyl-2-(methylsulfonyl) butanoate (3.26 g, 7.63 mmol), which may be produced as in Step B above, in tetrahydrofuran/methanol (4:1, 60 mL) at ambient temperature was added a solution of lithium hydroxide monohydrate (0.9 M in water, 15.3 mmol). The resulting mixture was stirred at ambient temperature for 3 hours. The mixture was acidified with aqueous hydrochloric acid (1N, 16 mL) and extracted three times with methylene chloride. The combined organic extracts were dried over magnesium sulfate, filtered and concentrated in vacuo to afford 4-(4-iodo-2-oxopyridin-1(2H)-yl)-2-methyl-2-(methylsulfonyl)butanoic acid as a solid. 3.05 g. LCMS: (M+1) 400.1

### Step D) Compound (VI) T3: 4-(4-iodo-2-oxopyridin-1(2H)-yl)-2-methyl-2-(methylsulfonyl)-N-(tetrahydro-2H-pyran-2-yloxy)butanamide

To a solution of 4-(4-iodo-2-oxopyridin-1(2H)-yl)-2-methyl-2-(methylsulfonyl)butanoic acid (3.01 g, 7.54 mmol), which may be produced as in Step C above, in methylene chloride (75 mL) at ambient temperature was added 1-(3-dimethylaminopropyl)-3-ethylcarbodiimide hydrochloride (2.02 g, 10.6 mmol), 1-hydroxy benzotriazole monohydrate (2.08 g, 13.6 mmol), triethyl amine (1.89 mL, 13.6 mmol) and O-tetrahydro-2H-pyran-2-yl-hydroxylamine (1.33 g, 11.3 mmol). The resulting mixture was stirred at ambient temperature overnight. The mixture was diluted with methylene chloride and water. The phases were separated and the aqueous extracted with methylene chloride two times. The organic extracts were combined and dried over magnesium sulfate, filtered and concentrated in vacuo to a crude residue. The crude residue was purified via silica gel chromatography eluting with methylene chloride and methanol. The fractions containing desired product were combined and concentrated to afford 4-(4-iodo-2-oxopyridin-1(2H)-yl)-2-methyl-2-(methylsulfonyl)-N-(tetrahydro-2H-pyran-2-yloxy)butanamide as a solid. 3.62 g. LCMS: (M-1) 497.

### Preparation 2B

### Synthesis of T6: (2R)-4-(4-iodo-2-oxopyridin-1(2H)-yl)-2-methyl-2-(methylsulfonyl)-N-(tetrahydro-2H-pyran-2-yloxy)butanamide

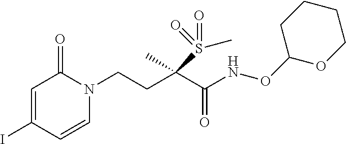 [SMILES: C[C@@](CCn1ccc(I)cc1=O)(C(=O)NOC1CCCCO1)S(C)(=O)=O]

### Step A) T4: Benzyl (2R)-4-(4-iodo-2-oxopyridin-1(2H)-yl)-2-methyl-2-(methylsulfonyl)butanoate

To a mixture of 4-iodopyridin-2(1H)-one which may be produced as in Step A of Preparation 2 (32.9 g, 149 mmol) and cesium carbonate (102 g, 312 mmol) in tetrahydrofuran (400 mL) at ambient temperature was added benzyl (2R)-4-bromo-2-methyl-2-(methylsulfonyl)butanoate (62.3 g, 178.4 mmol). The mixture was heated to 60° C. and stirred overnight. The mixture was allowed to cool to ambient temperature and filtered through a celite pad. The pad was washed with ethyl acetate (500 mL), the filtrates combined and concentrated in vacuo to afford an orange oil. The crude oil was purified via filtration through a silica gel pad, eluting with heptanes/ethyl acetate. The desired fractions were isolated and the solvent was removed via rotary evaporation affording benzyl (2R)-4-(4-iodo-2-oxopyridin-1(2H)-yl)-2-methyl-2-(methylsulfonyl) butanoate as a white solid. 44.91 g. ¹NMR (CDCl₃) δ ppm 7.39-7.36 (5H, m), 7.03 (1H, d, J=1.76 Hz), 6.77 (1H, d, J=7.03 Hz), 6.41 (1H, dd, J=1.76 Hz, J=7.03 Hz), 5.21 (2H, d, J=1.56 Hz), 4.19-4.12 (1H, m), 3.82-3.75 (1H, m), 2.97 (3H, s), 2.47-2.42 (2H, m), 1.73 (3H, s).

### Step B) T5: (2R)-4-(4-iodo-2-oxopyridin-1(2H)-yl)-2-methyl-2-(methylsulfonyl)butanoic Acid

To a solution of benzyl (2R)-4-(4-iodo-2-oxopyridin-1(2H)-yl)-2-methyl-2-(methylsulfonyl)butanoate (44.91 g, 91.7 mmol), which may be produced as in Step A above, in tetrahydrofuran (300 mL) and methanol (300 mL) at ambient temperature was added potassium hydroxide (3.76 M in water, 564 mmol). The resulting mixture was stirred at ambient temperature for 16 hours. The solvent was removed via via rotary evaporation and the residue was dissolved in water. The aqueous layer was washed with diethyl ether and then acidified with concentrated hydrochloric acid (˜pH 2) which afforded a white precipitate. The precipitate was collected via filtration, washed with water and dried in vacuo to a constant weight affording (2R)-4-(4-iodo-2-oxopyridin-1(2H)-yl)-2-methyl-2-(methylsulfonyl)butanoic acid as a white solid. 33.2 g. LCMS: (M⁺1) 400.4 ¹NMR (CD₃OD) δ ppm 7.34 (1H, d, J=7.23), 7.03 (1H, d, J=1.76), 6.69 (1H, dd, J=1.95, J=7.23), 4.24-4.16 (1H, m), 4.05-3.98 (1H, m), 3.14 (3H, s), 2.57-2.50 (1H, m), 2.35-2.28 (1H, m), 1.68 (3H, s).

### Step C) T6: (2R)-4-(4-iodo-2-oxopyridin-1(2H)-yl)-2-methyl-2-(methylsulfonyl)-N-(tetrahydro-2H-pyran-2-yloxy)butanamide

To a solution of (2R)-4-(4-iodo-2-oxopyridin-1(2H)-yl)-2-methyl-2-(methylsulfonyl) butanoic acid, which may be produced as in Step B above, (33.18 g, 83.12 mmol) in methylene chloride (400 mL) at ambient temperature was added 1-ethyl-3-(3-dimethylaminopropyl)carbodiimide hydrochloride (22.3 g, 116 mmol), 1-hydroxy benzotriazole monohydrate (22.9 g, 150 mmol), triethyl amine (20.9 mL, 150 mmol) and O-tetrahydro-2H-pyran-2-yl-hydroxylamine (14.6 g, 125 mmol). The resulting mixture was stirred at ambient temperature overnight. The mixture was diluted with methylene chloride and water. The phases separated and the aqueous extracted with methylene chloride two times. The organic extracts were combined and dried over magnesium sulfate, filtered and concentrated to a crude residue. The crude residue was dissolved in methylene chloride (˜150 mL) with minimal methanol. To this solution was added heptanes (450 mL) and the mixture was concentrated in vacuo to 150 mL and filtered. The solid was washed with heptanes and dried in vacuo to give (2R)-4-(4-iodo-2-oxopyridin-1(2H)-yl)-2-methyl-2-(methylsulfonyl)-N-(tetrahydro-2H-pyran-2-yloxy)butanamide. 26.1 g LCMS: (M-1) 497.6

### Preparation 3A: (2R)—N-hydroxy-2-methyl-2-(methylsulfonyl)-4-{2-oxo-4-[4-(2H-1,2,3-triazol-2-yl)phenyl] pyridin-1(2H)-yl}butanamide

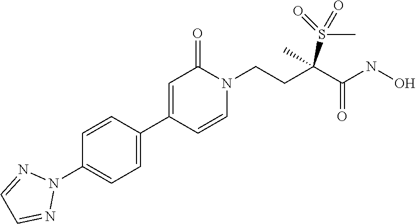

### Step A) Preparation of 2-[4-(4,4,5,5-Tetramethyl-1,3,2-dioxaborolan-2-yl)phenyl]-2H-1,2,3-triazole

Potassium acetate (391 mg, 3.98 mmol) was added to a solution of 2-(4-Bromophenyl)-2H-1,2,3-triazole (1.0 equivalent), 4,4,4′,4′,5,5,5′,5′-octamethyl-2,2′-bi-1,3,2-dioxaborolane (1.20 equivalents), and [1,1′-bis-(diphenylphosphino)ferrocene]-dichloropalladium (II) dcm complex (0.30 equivalents) in 1,4-dioxane in a vial. The vial was capped and heated to 80° C. and stirred at this temperature overnight. [1,1′-bis-(diphenylphosphino)ferrocene]-dichloropalladium (II) dcm complex (0.30 equivalents) was added to the reaction and the mixture was reheated to 80° C. and stirring was continued at this temperature overnight. The reaction was cooled, diluted with ethyl acetate and water, filtered through celite and the filter pad was washed with ethyl acetate. The organic layer was separated, and the aqueous layer was extracted with ethyl acetate. The combined organics were dried (MgSO₄), filtered, and concentrated. The crude was purified via flash chromatography using an Analogix SF15-24g column and ethyl acetate in heptane (30-80%) as the eluant to afford the title compound was converted to the title product. The title compound was obtained as an orange solid (240.6 mg, 78%) LC-MS m/z 272.4 (M⁺1). ¹H NMR (CDCl₃, 400 MHz) δ ppm 1.37 (s, 12H) 7.83 (s, 2H) 7.94 (d, J=8.59 Hz, 2H) 8.10 (d, J=8.59 Hz, 2H).

### Step B) (2R)-2-methyl-2-(methylsulfonyl)-4-{2-oxo-4-[4-(2H-1,2,3-triazol-2-yl)phenyl]pyridin-1(2H)-yl}-N-(tetrahydro-2H-pyran-2-yloxy)butanamide

Pd EnCat™ (0.08 equivalent) was added to a mixture of potassium carbonate (2.54 equivalent), 2-[4-(4,4,5,5-Tetramethyl-1,3,2-dioxaborolan-2-yl)phenyl]-2H-1,2,3-triazole (1.5 equivalents), and 4-(4-iodo-2-oxopyridin-1(2H)-yl)-2-methyl-2-(methylsulfonyl)-N-(tetrahydro-2H-pyran-2-yloxy)butanamide (1.0 equivalent) in dioxane:water (4:1) in a microwave vial and the reaction was heated at 90° C. overnight. The reaction was filtered and the resin was washed with ethyl acetate and water. The filtrate was concentrated to dryness and the crude was purified via flash chromatography on an Analogix SF15-12g column and eluted with ethyl acetate in heptane (0-80%) to afford the title compound. The title compound was obtained as a white solid (101 mg, 48.8%) LC-MS m/z 514.7 (M-1).

### Step C) (2R)—N-hydroxy-2-methyl-2-(methylsulfonyl)-4-{2-oxo-4-[4-(2H-1,2,3-triazol-2-yl)phenyl]pyridin-1(2H)-yl}butanamide

A 4.0 M solution of HCl in 1,4-dioxane was added slowly to a solution of (2R)-2-methyl-2-(methylsulfonyl)-4-{2-oxo-4-[4-(2H-1,2,3-triazol-2-yl)phenyl]pyridin-1(2H)-yl}-N-(tetrahydro-2H-pyran-2-yloxy)butanamide in dichloromethane with water (5:1) at 0° C. The ice bath was removed and the reaction was allowed to warm to rt. After 30 min (complete by TLC), the reaction was concentrated to afford a crude solid. The crude was triturated in isopropanol overnight. The solid was collected via filtration, washed with isopropanol, isopropanol:heptane (1:1), heptane, and ether. The title compound was obtained as an off-white solid (63.7 mg, 74%). LC-MS m/z 432.5 (M+1). ¹H NMR (400 MHz, DMSO-d₆) δ ppm 1.58 (s, 3H) 2.09-2.25 (m, 1H) 2.34-2.47 (m, 1H) 3.11 (s, 3H) 3.70-3.82 (m, 1H) 4.04-4.19 (m, 1H) 6.68-6.73 (m, 1H) 6.78 (d, J=2.15 Hz, 1H) 7.79 (d, J=7.22 Hz, 1H) 7.95 (d, J=8.78 Hz, 2H) 8.12 (d, J=8.59 Hz, 2H) 8.17 (s, 2H) 11.15 (br. s., 1H).

### Preparation 3B: (2R)-4-[4-(2,3-difluoro-4-methoxyphenyl)-2-oxopyridin-1(2H)-yl]-N-hydroxy-2-methyl-2-(methylsulfonyl)butanamide

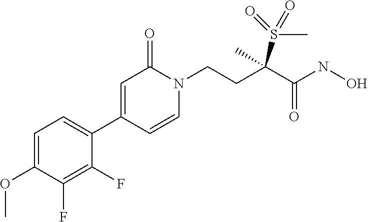

### Step A) (2R)-4-[4-(2,3-difluoro-4-methoxyphenyl)-2-oxopyridin-1(2H)-yl]-2-methyl-2-(methylsulfonyl)-N-(tetrahydro-2H-pyran-2-yloxy)butanamide

Pd EnCat™ (200 mg, 0.06 mmol) was added to a mixture of potassium carbonate (250 mg, 1.81 mmol), (2,3-difluoro-4-methoxyphenyl)boronic acid (113 mg, 0.602 mmol), and (2R)-4-(4-iodo-2-oxopyridin-1(2H)-yl)-2-methyl-2-(methylsulfonyl)-N-(tetrahydro-2H-pyran-2-yloxy)butanamide, (300 mg, 0.602 mmol) in dioxane:water (5.5 mL, 10:1 mixture) in a 25 mL round bottom flask. The flask was heated overnight at 80° C. The reaction was cooled to ambient temperature and filtered through celite and washed with ethyl acetate (20 mL). The crude material was concentrated to provide crude product. The resulting crude material was purified by chromatography on silica gel (elution solvent: ethyl acetate) to provide title compound as a viscous, foamy oil. Yield: 132 mg, 42.6%. MS (APCI) m/z 515.5 (M+H)¹H NMR (CDCl₃, 400 MHz) δ ppm 1.54-1.66 (m, 3H) 1.68 (d, J=2.34 Hz, 3H) 1.71-1.97 (m, 3H) 2.30-2.44 (m, 1H) 2.45-2.58 (m, 1H) 3.18 (d, J=3.12 Hz, 3H) 3.54-3.68 (m, 1H) 3.92 (s, 3H) 3.99-4.08 (m, 1H) 4.11-4.23 (m, 1H) 4.26-4.40 (m, 1H) 5.10-5.21 (m, 1H) 6.42-6.53 (m, 1H) 6.75 (s, 1H) 6.77-6.86 (m, 1H) 7.05-7.17 (m, 1H) 7.37 (d, J=7.02 Hz, 1H) 12.10 (d, J=7.61 Hz, 1H).

### Step B) (2R)-4-[4-(2,3-difluoro-4-methoxyphenyl)-2-oxopyridin-1(2H)-yl]-N-hydroxy-2-methyl-2-(methylsulfonyl)butanamide

A solution of 1.0 M aqueous HCl (2.76 mL) was added slowly to a solution of (2R)-4-[4-(2,3-difluoro-4-methoxyphenyl)-2-oxopyridin-1(2H)-yl]-2-methyl-2-(methylsulfonyl)-N-(tetrahydro-2H-pyran-2-yloxy)butanamide (132 mg, 0.26 mmol) in 1,4-dioxane (15 mL) at room temperature. The reaction was allowed to stir at room temperature overnight. After 18 hours the reaction was concentrated to 25% of the original volume, resulting in a white precipitate. The precipitate was filtered via Buchner funnel and washed with hexanes (20 mL) to afford a white solid. Yield 45 mg, 41%. MS (APCI) m/z 431.1 (M+H). ¹H NMR (400 MHz, DMSO-d₆) δ ppm 1.55 (s, 3H) 2.14 (td, J=12.20, 4.88 Hz, 1H) 2.35-2.45 (m, 1H) 3.08 (s, 3H) 3.72 (td, J=12.05, 4.78 Hz, 1H) 3.90 (s, 3H) 4.09 (td, J=11.90, 5.27 Hz, 1H) 6.46 (dt, J=7.02, 1.85 Hz, 1H) 6.54 (s, 1H) 7.03-7.17 (m, 1H) 7.37 (td, J=8.63, 2.24 Hz, 1H) 7.72 (d, J=7.22 Hz, 1H) 9.22 (br. s., 1H) 11.10 (s, 1H).

### Preparation 3C: (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamide

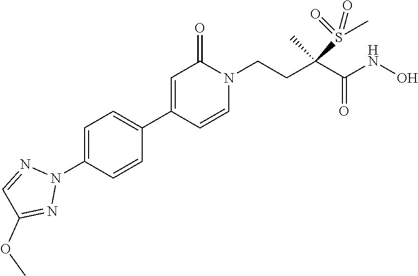 [SMILES: COc1cnn(-c2ccc(-c3ccn(CC[C@](C)(C(=O)NO)S(C)(=O)=O)c(=O)c3)cc2)n1]

### Step A) 2-(4-bromophenyl)-2H-1,2,3-triazole 1-oxide

Water (20 mL) was added to a flask containing glyoxal (2.0 g, 14 mmol). Hydroxylamine.HCl (958 mg, 13.8 mmol) and sodium carbonate (1.53 g, 14.5 mmol) were added in one portion to the glyoxal flask (CO₂ evolution observed). The reaction mixture was stirred at rt for 20 minutes (reaction mixture turned yellow). Methanol (40 mL) was added to the reaction mixture and 4-bromophenyl hydrazine.HCl (3.1 g, 13.8 mmol) was added portionwise under ice cooling. The reaction mixture was then stirred at rt for 30 min. Copper (II) sulfate.hexahydrate (20 g, 78 mmol) was was added to the reaction mixture. A water:pyridine (1:1) mixture (200 mL) was added then heated at 90° C. for 16 hours. The reaction mixture was cooled and adjusted to pH=3 with 6N HCl (approx 200 mL). The mixture was filtered through celite to remove insolubles. The celite was washed with additional ethyl acetate (1000 mL). The organic layer was separated and the product extracted additionally from the aqueous layer with EtOAc (3×250 mL). The organic phases were combined, dried over potassium carbonate, filtered and concentrated to approximately half the volume. This material was then filtered through a silica pad (approx 6 in.). Silica was washed with an additional 300 mL of ethyl acetate. The solvent was then concentrated in vacuo. The crude material was purified by chromatography on silica gel (4:1 heptane:EtOAc to 3:1 heptane:EtOAc). Concentrated fractions furnished a light tan solid (1.0 g, 30% TY). MS (LC/MS) m/z 240.1 (M+1). ¹H NMR (CDCl₃, 400 MHz) δ ppm 7.47 (d, J=0.98 Hz, 1H) 7.65-7.69 (m, 2H) 7.73 (d, J=0.78 Hz, 1H) 7.86-7.90 (m, 2H)

### Step B) 2-(4-bromophenyl)-2H-1,2,3-triazol-4-yl acetate

Acetyl chloride (4.71 ml, 63 mmol) was added to a flask containing 2-(4-bromophenyl)-2H-1,2,3-triazole 1-oxide (500 mg, 2.08 mmol) and was stirred at rt for 16 hours. Acetyl chloride was removed in vacuo and ethyl acetate (30 mL) was added and concentrated (2×) to furnish a light brown solid (520 mg, 90%). MS (LC/MS) m/z 282.1 (M+1). ¹H NMR (CDCl₃, 400 MHz) δ ppm 2.39 (s, 3H) 7.57-7.63 (m, 2H) 7.84 (s, 1H) 7.87-7.93 (m, 2H).

### Step C) 2-(4-bromophenyl)-2H-1,2,3-triazol-4-ol

2-(4-bromophenyl)-2H-1,2,3-triazol-4-yl acetate (520 mg, 1.84 mmol) was treated with methanol (10 mL) and water (10 mL) followed by 1,4-dioxane (5 mL). The resulting solution was treated with lithium hydroxide (265 mg, 11.1 mmol). The reaction mixture was stirred at rt for 36 hours. 1N HCl (40 mL) was added to the reaction mixture and the product was extracted with ethyl acetate (3×100 mL). The combined organic phases were dried over potassium carbonate, filtered, and concentrated. The crude material was purified by chromatography on silica gel (4:1 heptane:EtOAc 1:4 heptane:EtOAc) to furnish a light tan solid (440 mg, 98% TY). MS (LC/MS) m/z 240.21 (M+1). ¹H NMR (CDCl₃, 400 MHz) δ ppm 7.33 (s, 1H) 7.58 (d, J=8.98 Hz, 2H) 7.78 (d, J=8.98 Hz, 2H).

### Step D) 2-(4-bromophenyl)-4-methoxy-2H-1,2,3-triazole

2-(4-bromophenyl)-2H-1,2,3-triazol-4-ol (200 mg, 0.833 mmol) was weighed into a 20 mL vial equipped with a septa cap. THE (10.0 mL) was added. To this was added cesium carbonate (814 mg, 2.5 mmol), followed by the addition of methyl iodide (65.8 uL, 1.04 mmol) via syringe. The reaction was heated at 60° C. for 16 hours. Water (20 mL) was added and the product was extracted with ethyl acetate (2×75 mL). Organic phases were combined, dried over potassium carbonate, filtered and concentrated to furnish a light tan solid (190 mg, 89% TY). ¹H NMR (CDCl₃, 400 MHz) δ ppm 4.04 (s, 3H) 7.30 (s, 1H) 7.56 (d, J=8.98 Hz, 2H) 7.84 (d, J=8.98 Hz, 2H).

### Step E) 4-methoxy-2-[4-(4,4,5,5-tetramethyl-1,3,2-dioxaborolan-2-yl)phenyl]-2H-1,2,3-triazole

Potassium acetate (220 mg, 2.24 mmol) was added to 2-(4-bromophenyl)-4-methoxy-2H-1,2,3-triazole (190 mg, 0.748 mmol), bis(pinacolato)diboron (228 mg, 0.898 mmol) and Pd(dppf)Cl₂.DCM complex (185 mg, 0.224 mmol) in a 20 mL vial equipped with a septa cap. The vial was evacuated and backfilled with nitrogen 3×. To this was added 1,4-dioxane (8 mL). The reaction mixture was heated at 80° C. for 16 hours. The reaction mixture was filtered through celite (approx 2 inches). The celite was washed with additional ethyl acetate (150 mL). The filtrate was concentrated in vacuo and the crude material was purified by chromatography on silica gel (9:1 heptane:EtOAc to 2:4 heptane:EtOAc) to furnish a light tan solid (145 mg, 65% TY). MS (LC/MS) m/z 302.3 (M+1). ¹H NMR (CDCl₃, 400 MHz) δ ppm 1.37 (s, 12H) 4.06 (s, 3H) 7.31 (s, 1H) 7.90 (s, 2H) 7.95 (s, 2H).

### Step F) (2R)-4-{4-[4-(4-methoxy-2H-1,2,3-triazol-2-yl)phenyl]-2-oxopyridin-1(2H)-yl}-2-methyl-2-(methylsulfonyl)-N-(tetrahydro-2H-pyran-2-yloxy)butanamide

Pd EnCat™ (98 mg, 0.03 mmol) was added to a mixture of potassium carbonate (171 mg, 1.24 mmol), 4-methoxy-2-[4-(4,4,5,5-tetramethyl-1,3,2-dioxaborolan-2-yl)phenyl]-2H-1,2,3-triazole (138 mg, 0.457 mmol) and (2R)-4-(4-iodo-2-oxopyridin-1(2H)-yl)-2-methyl-2-(methylsulfonyl)-N-(tetrahydro-2H-pyran-2-yloxy)butanamide (190 mg, 0.381 mmol) in dioxane:water (6 mL, 5:1) in a 20 mL vial. The reaction was cooled and filtered through celite (approx 1 inch). The celite was washed with additional methanol (100 mL). The filtrate was concentrated in vacuo and the crude material was purified by chromatography on silica gel (4:1 heptane:EtOAc to 100% EtOAc to 85% EtOAc:15% methanol) to furnish alight tan gum (120 mg, 58% TY). MS (LC/MS) m/z 546.2 (M⁺1). ¹H NMR (CD₃OD, 400 MHz) δ ppm 1.28 (s, 1H) 1.57-1.70 (m, 2H) 1.68-1.81 (m, 3H) 1.78-1.92 (m, 3H) 2.36-2.50 (m, 1H) 2.55-2.72 (m, 1H) 3.09-3.21 (m, 3H) 3.56-3.70 (m, 1H) 4.07 (s, 3H) 4.12 (d, J=7.22 Hz, 2H) 4.15-4.25 (m, 1H) 4.25-4.42 (m, 1H) 5.01-5.14 (m, 1H) 6.76-6.85 (m, 1H) 6.87 (s, 1H) 7.49 (s, 1H) 7.68-7.80 (m, 1H) 7.85 (d, J=9.17 Hz, 2H) 8.08 (d, J=8.98 Hz, 2H)

### Step G) (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamide

To (2R)-4-{4-[4-(4-methoxy-2H-1,2,3-triazol-2-yl)phenyl]-2-oxopyridin-1(2H)-yl}-2-methyl-2-(methylsulfonyl)-N-(tetrahydro-2H-pyran-2-yloxy)butanamide (120 mg, 0.22 mmol) was added dioxane (2 mL), dichloromethane (2 mL), and water (1 mL). The reaction flask was cooled externally with ice then treated with a 4.0 M HCl in dioxane (0.55 mL). The reaction mixture was stirred for 15 minutes then concentrated under reduced pressure. Isopropanol (10 mL) was added and concentrated to azeotrope any remaining water to furnish a tan solid (80 mg, 80% TY). MS (LC/MS) m/z 462.3 (M⁺1). ¹H NMR (CD₃OD, 400 MHz) δ ppm 1.74 (s, 3H) 2.34-2.51 (m, 1H) 2.55-2.81 (m, 1H) 3.13 (s, 3H) 3.96-4.06 (m, 1H) 4.07 (s, 3H) 4.26-4.45 (m, 1H) 6.84-7.00 (m, 2H) 7.49 (s, 1H) 7.75-7.93 (m, 3H) 8.09 (d, J=8.78 Hz, 2H).

### Preparation 3D: (2R)—N-hydroxy-2-methyl-2-(methylsulfonyl)-4-{2-oxo-4-[4-(1,3-thiazol-2-yl)phenyl]pyridin-1(2H)-yl}butanamide

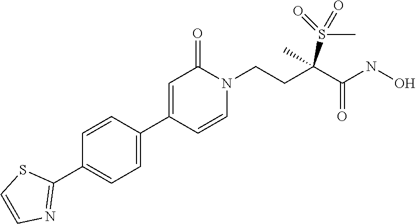

The title compound can be made in a manner analogous to the procedures described hereinabove. The product can typically be derived from a Suzuki-Miyaura cross coupling with optional deprotection of a terminal hydroxamic acid protecting group. Methods used to describe the synthesis of the precursors or coupling partners such as boronic acids or esters are known to those skilled in the art. Retention time: 0.48 Mass ion 448. ¹H NMR (400 MHz, DMSO-d₆) δ ppm 1.58 (s, 3H) 2.18 (td, J=12.05, 4.98 Hz, 1H) 2.40-2.48 (m, 1H) 3.11 (s, 3H) 3.77 (td, J=12.15, 5.37 Hz, 1H) 4.08-4.19 (m, 1H) 6.72 (dd, J=7.22, 2.15 Hz, 1H) 6.79 (d, J=2.15 Hz, 1H) 7.80 (d, J=7.22 Hz, 1H) 7.84.

### Preparation 4A: (R)-4-(4-(4-(2H-1,2,3-triazol-2-yl)phenyl)-6-oxopyrimidin-1(6H)-yl)-N-hydroxy-2-methyl-2-(methylsulfonyl)butanamide

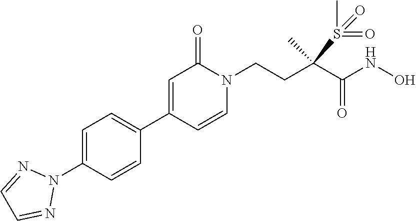 [SMILES: C[C@@](CCn1ccc(-c2ccc(-n3nccn3)cc2)cc1=O)(C(=O)NO)S(C)(=O)=O]

### Step A: Preparation of 4-(4-(2H-1,2,3-triazol-2-yl)phenyl)-6-methoxypyrimidine

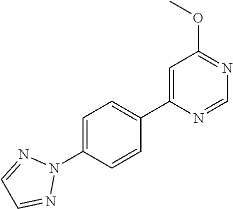 [SMILES: COc1cc(-c2ccc(-n3nccn3)cc2)ncn1]

The following reaction was carried out on the same scale in two separate runs with the difference between the runs being the heating method and heating time. To a mixture of 2-(4-(4,4,5,5-tetramethyl-1,3,2-dioxaborolan-2-yl)phenyl)-2H-1,2,3-triazole (619 mg, 2.28 mmol) and 4-chloro-6-methoxypyrimidine (300 mg, 2.08 mmol) was added bis(triphenylphosphine)palladium (II) chloride (150 mg, 0.21 mmol) then 1,2-dimethoxyethane (6 mL), ethanol (2 mL) and 2.0 M aqueous sodium carbonate (3.1 mL). The reaction mixture was either heated at 120° C. in a microwave for 15 minutes or alternatively was heated in an oil bath at 120° C. for 1 hour. The reaction mixture was purified by flash chromatography on silica gel using gradient elution (heptane:EtOAc, 0˜100%). The product containing fractions were concentrated in vacuo to provide the title compound (130 mg, 25% yield from microwave heating; 80 mg, 15% yield from oil bath heating). ¹H NMR (400 MHz, CDCl₃) δ ppm 8.86-8.90 (m, 1H), 8.21 (m, 4H), 7.87 (s, 2H), 7.15-7.18 (m, 1H), 4.06 (s, 3H).

### Step B: Preparation of 6-(4-(2H-1,2,3-triazol-2-yl)phenyl)pyrimidin-4(3H)-one

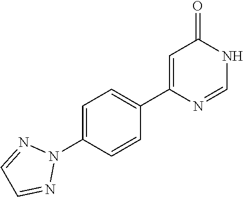 [SMILES: O=c1cc(-c2ccc(-n3nccn3)cc2)nc[nH]1]

To a solution of 4-(4-(2H-1,2,3-triazol-2-yl)phenyl)-6-methoxypyrimidine (210 mg, 0.829 mmol) in acetic acid (6 mL) was added hydrobromic acid (0.533 mL). The reaction mixture was heated overnight at 85° C. then was concentrated in vacuo. EtOAc was added to the residue then the mixture was concentrated in vacuo to provide the title compound. The product was used in the next step.

### Step C: Preparation of ethyl (R)-4-(4-(4-(2H-1,2,3-triazol-2-yl)phenyl)-6-oxopyrimidin-1(6H)-yl)-2-methyl-2-(methylsulfonyl)butanoate

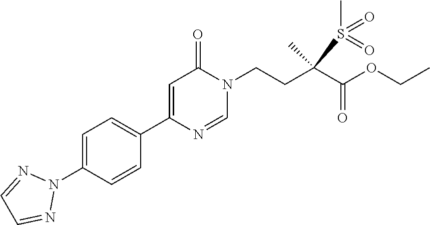 [SMILES: CCOC(=O)[C@@](C)(CCn1cnc(-c2ccc(-n3nccn3)cc2)cc1=O)S(C)(=O)=O]

A suspension of 6-(4-(2H-1,2,3-triazol-2-yl)phenyl)pyrimidin-4(3H)-one (260 mg, 1.09 mmol), ethyl (R)-4-bromo-2-methyl-2-(methylsulfonyl)butanoate (344 mg, 1.20 mmol), potassium carbonate (451 mg, 3.26 mmol) and tetrabutyl ammonium bromide (35.9 mg, 0.11 mmol) in acetonitrile (10 mL) was refluxed for 1 hour. A white precipitate had formed and LC/MS indicated no product so additional acetonitrile was added (10 mL). The reaction mixture was refluxed overnight. LC/MS indicates a mixture of two products had formed (O-alkylation and N-alkylation products). The reaction mixture was allowed to cool then concentrated in vacuo. The residue was filtered through a small silica gel column eluted with methylene chloride and the filtrate was concentrated in vacuo. The resulting residue was then purified by flash chromatography on silica gel using gradient elution (heptane:EtOAc, 40˜100% EtOAc). The first product (O-alkylated eluted in 50% heptane/EtOAc while the second product (desired N-alkylated) eluted in 20% heptane/80% EtOAc. The product containing fractions were concentrated in vacuo to provide the title compound (160 mg, 33% yield).

### Step D: Preparation of (R)-4-(4-(4-(2H-1,2,3-triazol-2-yl)phenyl)-6-oxopyrimidin-1(6H)-yl)-2-methyl-2-(methylsulfonyl)butanoic Acid

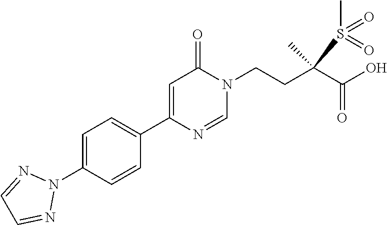 [SMILES: C[C@@](CCn1cnc(-c2ccc(-n3nccn3)cc2)cc1=O)(C(=O)O)S(C)(=O)=O]

To a solution of ethyl (R)-4-(4-(4-(2H-1,2,3-triazol-2-yl)phenyl)-6-oxopyrimidin-1(6H)-yl)-2-methyl-2-(methylsulfonyl)butanoate (160 mg, 0.359 mmol) in 2-methyltetrahydrofuran (5 mL) was added a solution of lithium hydroxide (43.0 mg, 1.80 mmol). The reaction mixture was heated at 50° C. overnight and LC/MS indicates that the product was formed. The mixture was allowed to cool then the layers were separated. The organic layer was treated with 1N sodium hydroxide (4 mL). The combined aqueous layer was acidified to pH 2 with 3N hydrochloric acid. A white creamy solid formed and was collected by filtration and dried to provide the title compound (100 mg, 67% yield).

### Step E: Preparation of (2R)-4-(4-(4-(2H-1,2,3-triazol-2-yl)phenyl)-6-oxopyrimidin-1(6H)-yl)-2-methyl-2-(methylsulfonyl)-N-((tetrahydro-2H-pyran-2-yl)oxy)butanamide

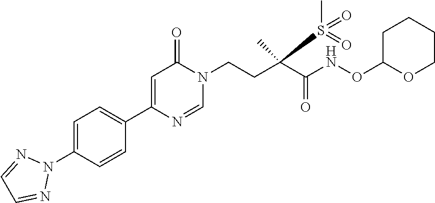 [SMILES: C[C@@](CCn1cnc(-c2ccc(-n3nccn3)cc2)cc1=O)(C(=O)NOC1CCCCO1)S(C)(=O)=O]

To a suspension of (R)-4-(4-(4-(2H-1,2,3-triazol-2-yl)phenyl)-6-oxopyrimidin-1(6H)-yl)-2-methyl-2-(methylsulfonyl)butanoic acid (100 mg, 0.24 mmol) in 2-methyltetrahydrofuran (5 mL) was added N-methylmorpholine (0.04 mL, 0.36 mmol) and 2-chloro-4,6-dimethoxy-1,3,5-triazine (56.5 mg, 0.312 mmol). The reaction mixture was stirred for one hour at RT then O-(tetrahydro-2H-pyran-2-yl)hydroxylamine (36.6 mg, 0.312 mmol) was added and the reaction mixture was stirred for 1 hour at RT. The reaction mixture was then filtered and concentrated in vacuo. The residue was dissolved in dichloromethane and the resulting solution was then purified by flash chromatography on silica gel using gradient elution (heptane:EtOAc, 40˜100% EtOAc). The product containing fractions which eluted in 50% EtOAc/50% heptane were concentrated in vacuo to provide the title compound (50 mg, 40%).

### Step F: Preparation of (R)-4-(4-(4-(2H-1,2,3-triazol-2-yl)phenyl)-6-oxopyrimidin-1(6H)-yl)-N-hydroxy-2-methyl-2-(methylsulfonyl)butanamide

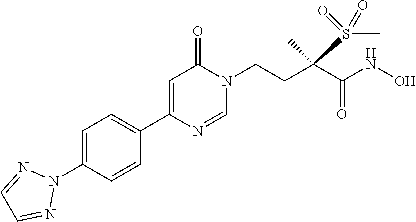 [SMILES: C[C@@](CCn1cnc(-c2ccc(-n3nccn3)cc2)cc1=O)(C(=O)NO)S(C)(=O)=O]

To a solution of (2R)-4-(4-(4-(2H-1,2,3-triazol-2-yl)phenyl)-6-oxopyrimidin-1(6H)-yl)-2-methyl-2-(methylsulfonyl)-N-((tetrahydro-2H-pyran-2-yl)oxy)butanamide (50.0 mg, 0.10 mmol) in dioxane (5 mL) was added hydrogen chloride (0.50 mmol, 0.125 mL of 4.0 M in diethyl ether). The reaction mixture was stirred for one hour then was concentrated in vacuo and the residue was washed with ethyl acetate and ethanol to provide the title compound (40 mg, 93%). ¹H NMR (400 MHz, DMSO-d₆) δ ppm 1.59 (s, 3H), 2.20 (ddd, J=13.22, 11.07, 4.98 Hz, 1H), 2.52-2.58 (m, 1H), 3.10 (s, 3H), 3.84 (ddd, J=12.93, 10.88, 5.27 Hz, 1H), 4.09 (ddd, J=12.93, 10.88, 4.68 Hz, 1H), 7.04-7.07 (m, 1H), 8.11-8.16 (m, 2H), 8.19 (s, 2H), 8.26-8.31 (m, 2H), 8.52-8.64 (m, 1H).

### Example 1

### (R)-4-(4-(4-(2H-1,2,3-triazol-2-yl)phenyl)-2-oxopyridin-1(2H)-yl)-2-methyl-2-(methylsulfonyl)butanamido Phosphate, Disodium Salt

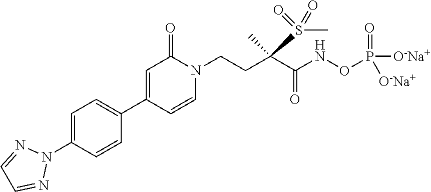

(R)-4-(4-(4-(2H-1,2,3-Triazol-2-yl)phenyl)-2-oxopyridin-1(2H)-yl)-N-hydroxy-2-methyl-2-(methylsulfonyl)butanamide (500 mg, 1.16 mmol) was heated in tetrahydrofuran (150 mL) until it dissolved, whereupon it was cooled to RT and triethylamine (3.9 mL, 28 mmol) was added. The mixture was then cooled to −40° C. and phosphorus oxychloride (0.32 mL, 3.3 mmol) was added and the mixture warmed to −12° C. and water (20 mL) was added. The mixture was allowed to warm to room temperature and stirred overnight. The solution was then extracted with ethyl acetate and the combined organic extracts extracted with water. The combined water layers were then partially evaporated and 4M NaOH was added until pH=13 and the aqueous layer evaporated to give an off-white solid. A 1:1 mixture of DMSO and water was added and was decanted before the white solid was triturated with water (10 mL) to give a white solid. ¹H-NMR (400 MHz, D₂O) δ 1.6 (s, 3H), 2.25 (dt, 1H), 2.6 (dt, 1H), 3.25 (s, 3H), 4.00 (dt, 1H), 4.25 (dt, 1H), 6.75 (s, 1H), 6.85 (d, 1H), 7.75 (d, 2H), 7.85 (d, 1H), 7.9 (d, 2H), 8.00 (s, 2H). m/z (Cl) 512 (M-2Na+3H).

### Example 2

### (2R)-4-[4-(2,3-difluoro-4-methoxyphenyl)-2-oxopyridin-1(2H)-yl]-N-hydroxy-2-methyl-2-(methylsulfonyl)butanamido Phosphate, Disodium Salt

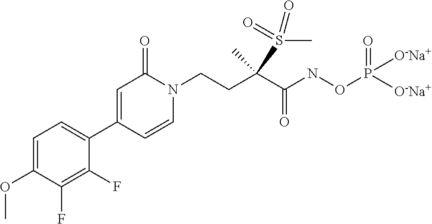

The title compound can be prepared using the procedure as described for Example 1 by using (2R)-4-[4-(2,3-difluoro-4-methoxyphenyl)-2-oxopyridin-1(2H)-yl]-N-hydroxy-2-methyl-2-(methylsulfonyl)butanamide as the starting material.

### Example 3

### (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido Phosphate, Disodium Salt

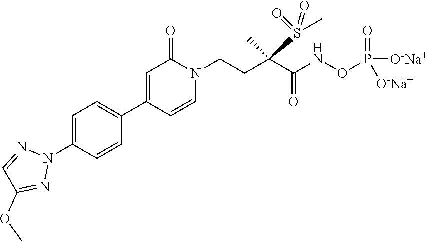

The title compound can be prepared using the procedure as described for Example 1 by using (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamide as the starting material.

### Example 4

### (2R)—N-hydroxy-2-methyl-2-(methylsulfonyl)-4-{2-oxo-4-[4-(1,3-thiazol-2-yl)phenyl]pyridin-1(2H)-yl}butanamido Phosphate, Disodium Salt

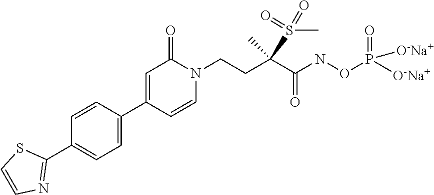

The title compound can be prepared using the procedure as described for Example 1 by using (2R)—N-hydroxy-2-methyl-2-(methylsulfonyl)-4-{2-oxo-4-[4-(1,3-thiazol-2-yl)phenyl]pyridin-1(2H)-yl}butanamide as the starting material.

### Example 5

### (R)-4-(4-(4-(2H-1,2,3-triazol-2-yl)phenyl)-6-oxopyrimidin-1(6H)-yl)-N-hydroxy-2-methyl-2-(methylsulfonyl)butanamido Phosphate, Disodium Salt

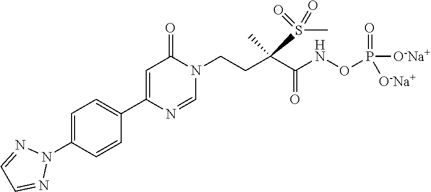

The title compound can be prepared using the procedure as described for Example 1 by using (R)-4-(4-(4-(2H-1,2,3-triazol-2-yl)phenyl)-6-oxopyrimidin-1(6H)-yl)-N-hydroxy-2-methyl-2-(methylsulfonyl)butanamide as starting material.

### Example 6

### (R)-4-(4-(4-(2H-1,2,3-triazol-2-yl)phenyl)-2-oxopyridin-1(2H)-yl)-2-methyl-2-(methylsulfonyl)butanamido Phosphate, Diammonium Salt

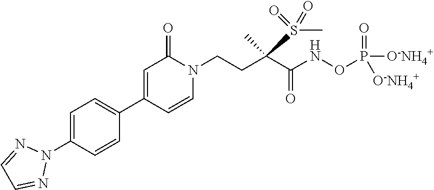

The title compound can be prepared in a manner analogous to the compound of Example 1 using concentrated aqueous ammonium hydroxide instead of the 4M NaOH.

### Example 7

### (2R)-4-[4-(2,3-difluoro-4-methoxyphenyl)-2-oxopyridin-1(2H)-yl]-N-hydroxy-2-methyl-2-(methylsulfonyl)butanamido Phosphate, Diammonium Salt

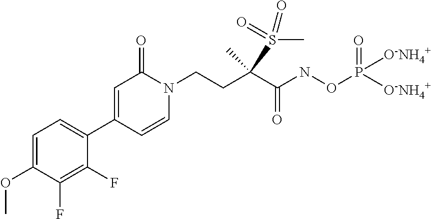

The title compound can be prepared in a manner analogous to the compound of Example 2 using concentrated aqueous ammonium hydroxide instead of the 4M NaOH.

### Example 8

### (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido Phosphate, Diammonium Salt

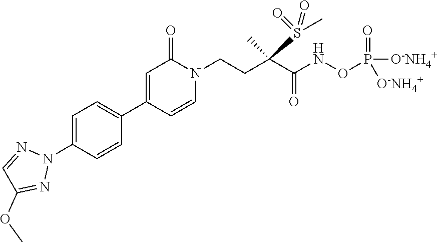

The title compound can be prepared in a manner analogous to the compound of Example 3 using concentrated aqueous ammonium hydroxide instead of the 4M NaOH.

### Example 9

### (2R)—N-hydroxy-2-methyl-2-(methylsulfonyl)-4-{2-oxo-4-[4-(1,3-thiazol-2-yl)phenyl]pyridin-1(2H)-yl}butanamido Phosphate, Ammonium Salt

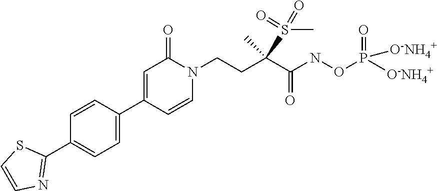

The title compound can be prepared in a manner analogous to the compound of Example 3 using concentrated aqueous ammonium hydroxide instead of the 4M NaOH.

### Example 10

### (R)-4-(4-(4-(2H-1,2,3-triazol-2-yl)phenyl)-6-oxopyrimidin-1(6H)-yl)-N-hydroxy-2-methyl-2-(methylsulfonyl)butanamido Phosphate, Diammonium Salt

### Examples 11-15

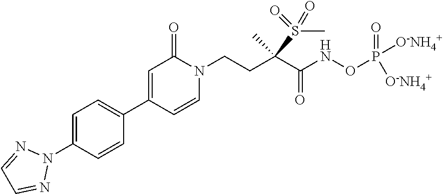

Examples 11-15 can be prepared in a manner analogous to the corresponding compounds of Examples 1-5 using 4M KOH instead of the 4M NaOH.

Example 11: (R)-4-(4-(4-(2H-1,2,3-triazol-2-yl)phenyl)-2-oxopyridin-1(2H)-yl)-2-methyl-2-(methylsulfonyl)butanamido phosphate, dipotassium salt.

Example 12: (2R)-4-[4-(2,3-difluoro-4-methoxyphenyl)-2-oxopyridin-1(2H)-yl]-N-hydroxy-2-methyl-2-(methylsulfonyl)butanamido phosphate, dipotassium salt.

Example 13: (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, dipotassium salt.

Example 14: (2R)—N-hydroxy-2-methyl-2-(methylsulfonyl)-4-{2-oxo-4-[4-(1,3-thiazol-2-yl)phenyl]pyridin-1(2H)-yl}butanamido phosphate, dipotassium salt.

Example 15: (R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate, dipotassium salt.

### Examples 16-20

Examples 16-20 can be prepared in a manner analogous to the corresponding compounds of Examples 1-5 using 4M LiH instead of the 4M NaOH.

Example 16: (R)-4-(4-(4-(2H-1,2,3-triazol-2-yl)phenyl)-2-oxopyridin-1(2H)-yl)-2-methyl-2-(methylsulfonyl)butanamido phosphate, dilithium salt.

Example 17: (2R)-4-[4-(2,3-difluoro-4-methoxyphenyl)-2-oxopyridin-1(2H)-yl]-N-hydroxy-2-methyl-2-(methylsulfonyl)butanamido phosphate, dilithium salt.

Example 18: (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, dilithium salt.

Example 19: (2R)—N-hydroxy-2-methyl-2-(methylsulfonyl)-4-{2-oxo-4-[4-(1,3-thiazol-2-yl)phenyl]pyridin-1(2H)-yl}butanamido phosphate, dilithium salt.

Example 20: (R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate, dilithium salt.

### General Procedure I for the Preparation of Salts

Dowex-50wx8-100 cation exchange resin is washed with water, methanol, and water again. The resin is then basified by treatment with an appropriate metal hydroxide (such as lithium hydroxide, potassium hydroxide, sodium hydroxide), ammonium hydroxide, amino acid or organic amine solution and is then washed with water. Divide the resin which is ready to use into three portions. To a solution of the appropriate pyridinone or pyrimidinone hydroxamic acid phosphate salt (such as a ammonium or diammonium salt or the sodium or disodium salt (e.g. a compound of Example 1-10 or a corresponding mono salt)) in water is added one portion of the resin. Stir the mixture for 10 minutes then filter it and rinse the solid with water. Add another portion of the resin to the combined filtrate and stir for 10 minutes, filter and rinse the solid with water. Add the final portion of resin, stir for 10 minutes, filter and rinse the solid with water. Concentrate the filtrate in vacuo, dissolve the residue in acetonitrile, filter, and concentrate the filtrate in vacuo. Dissolve the residue is methylene chloride, add hexane and concentrate in vacuo to provide the corresponding phosphate mono or di salt.

### General Procedure II for the Preparation of Divalent Cation Salts

One equivalent of an appropriate pyridinone or pyrimidinone hydroxamic acid phosphate (such as (R)-4-(4-(4-(2H-1,2,3-triazol-2-yl)phenyl)-2-oxopyridin-1(2H)-yl)-2-methyl-2-(methylsulfonyl)butanamido phosphate, (2R)-4-[4-(2,3-difluoro-4-methoxyphenyl)-2-oxopyridin-1(2H)-yl]-N-hydroxy-2-methyl-2-(methylsulfonyl) butanamido phosphate, (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, (2R)—N-hydroxy-2-methyl-2-(methylsulfonyl)-4-{2-oxo-4-[4-(1,3-thiazol-2-yl)phenyl]pyridin-1(2H)-yl}butanamido phosphate, or (R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate) is taken up in an appropriate solvent such as methanol at a concentration of approximately 10 mg/mL and is treated with one equivalent of the corresponding metal acetate (such as calcium acetate, zinc acetate or magnesium acetate). The resulting mixture is stirred at ambient temperature for several days then is concentrated in vacuo. The resulting residue is washed with a small amount of methanol and the product is dried.

The following Examples 21-23 can be prepared according to General Procedure II.
Example 21: (R)-4-(4-(4-(2H-1,2,3-triazol-2-yl)phenyl)-2-oxopyridin-1(2H)-yl)-2-methyl-2-(methylsulfonyl)butanamido phosphate, calcium salt.
Example 22: (R)-4-(4-(4-(2H-1,2,3-triazol-2-yl)phenyl)-2-oxopyridin-1(2H)-yl)-2-methyl-2-(methylsulfonyl)butanamido phosphate, magnesium salt.
Example 23: (R)-4-(4-(4-(2H-1,2,3-triazol-2-yl)phenyl)-2-oxopyridin-1(2H)-yl)-2-methyl-2-(methylsulfonyl)butanamido phosphate, zinc salt.

### General Procedure III for the Preparation of Monovalent Cation Salts

One equivalent of an appropriate pyridinone or pyrimidinone hydroxamic acid phosphate (such as (R)-4-(4-(4-(2H-1,2,3-triazol-2-yl)phenyl)-2-oxopyridin-1(2H)-yl)-2-methyl-2-(methylsulfonyl)butanamido phosphate, (2R)-4-[4-(2,3-difluoro-4-methoxyphenyl)-2-oxopyridin-1(2H)-yl]-N-hydroxy-2-methyl-2-(methylsulfonyl) butanamido phosphate, (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamido phosphate, (2R)—N-hydroxy-2-methyl-2-(methylsulfonyl)-4-{2-oxo-4-[4-(1,3-thiazol-2-yl)phenyl]pyridin-1(2H)-yl}butanamido phosphate, or (R)-4-(4-(4-(2H-1,2,3-triazol-2-yl)phenyl)-6-oxopyrimidin-1(6H)-yl)-N-hydroxy-2-methyl-2-(methylsulfonyl)butanamido phosphate) is taken up in an appropriate solvent such as methanol at a concentration of approximately 10 mg/mL and is treated with 1.0 to 1.1 equivalents of the appropriate corresponding amine (such as pyrrolidine, piperidine, pyridine, morpholine, piperazine, tris-(hydroxymethyl)methylamine, diethylamine, glycine). The resulting mixture is stirred at ambient temperature for several days then is concentrated in vacuo. The resulting residue is washed with a small amount of methanol and the product is dried.

The following Examples 24-27 can be prepared according to General Procedure III
Example 24: (R)-4-(4-(4-(2H-1,2,3-triazol-2-yl)phenyl)-2-oxopyridin-1(2H)-yl)-2-methyl-2-(methylsulfonyl)butanamido phosphate, pyrrolidine salt.
Example 25: (R)-4-(4-(4-(2H-1,2,3-triazol-2-yl)phenyl)-2-oxopyridin-1(2H)-yl)-2-methyl-2-(methylsulfonyl)butanamido phosphate, tris-(hydroxymethyl)methylamine Salt.
Example 26: (R)-4-(4-(4-(2H-1,2,3-triazol-2-yl)phenyl)-2-oxopyridin-1(2H)-yl)-2-methyl-2-(methylsulfonyl)butanamido phosphate, diethylamine salt.
Example 27: (R)-4-(4-(4-(2H-1,2,3-triazol-2-yl)phenyl)-2-oxopyridin-1(2H)-yl)-2-methyl-2-(methylsulfonyl)butanamido Phosphate, Glycine Salt.

### General Procedure IV For Preparation of Boronates

One equivalent of an appropriate hydroxamic acid (e.g. (R)-4-(4-(4-(2H-1,2,3-Triazol-2-yl)phenyl)-2-oxopyridin-1(2H)-yl)-N-hydroxy-2-methyl-2-(methylsulfonyl) butanamide, (2R)-4-[4-(2,3-difluoro-4-methoxyphenyl)-2-oxopyridin-1(2H)-yl]-N-hydroxy-2-methyl-2-(methylsulfonyl)butanamide, (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamide, (2R)—N-hydroxy-2-methyl-2-(methylsulfonyl)-4-{2-oxo-4-[4-(1,3-thiazol-2-yl)phenyl]pyridin-1(2H)-yl}butanamide, or (R)-4-(4-(4-(2H-1,2,3-triazol-2-yl)phenyl)-6-oxopyrimidin-1(6H)-yl)-N-hydroxy-2-methyl-2-(methylsulfonyl)butanamide) is suspended in water (at a concentration of approximately 1.5 M). Boric acid (1.0 equivalent) is added, followed by an appropriate base (e.g. sodium hydroxide, potassium hydroxide or lithium hydroxide (1.0 equivalent)). The reaction is allowed to stir at room temperature for approximately 30 minutes. The reaction solution is filtered via a teflon filter. The filtrate is transferred to a 250 mL round-bottom flask, where it is frozen at −78° C. The frozen solid is placed on a lyophilizer and is allowed to dry overnight (vacuum=0.2 mbar) to provide the desired product.

### Example 28

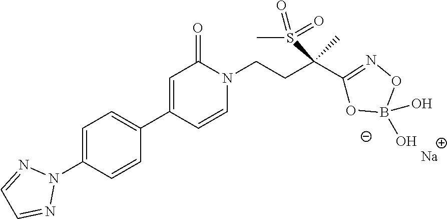

The compound shown above, sodium (R)-5-(4-(4-(4-(2H-1,2,3-triazol-2-yl)phenyl)-2-oxopyridin-1(2H)-yl)-2-(methylsulfonyl)butan-2-yl)-2,2-dihydroxy-1,3,4,2-dioxazaborol-2-uide, can be prepared according to General Procedure IV using (R)-4-(4-(4-(2H-1,2,3-Triazol-2-yl)phenyl)-2-oxopyridin-1(2H)-yl)-N-hydroxy-2-methyl-2-(methylsulfonyl) butanamide as starting material and sodium hydroxide as the base.

### Example 29

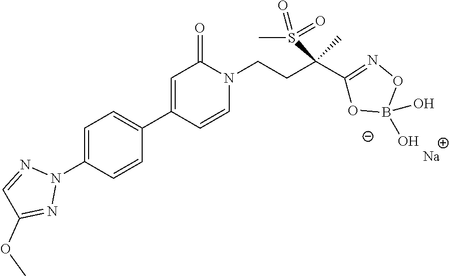

The compound shown above, sodium (R)-5-(4-(4-(2,3-difluoro-4-methoxyphenyl)-2-oxopyridin-1(2H)-yl)-2-(methylsulfonyl)butan-2-yl)-2,2-dihydroxy-1,3,4,2-dioxazaborol-2-uide, can be prepared according to General Procedure IV using (2R)-4-[4-(2,3-difluoro-4-methoxyphenyl)-2-oxopyridin-1(2H)-yl]-N-hydroxy-2-methyl-2-(methylsulfonyl)butanamide as starting material and sodium hydroxide as the base.

### Example 30

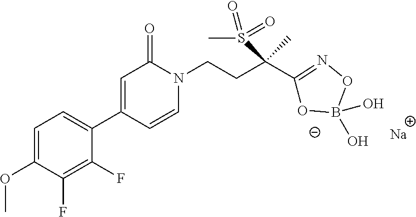

The compound shown above, sodium (R)-2,2-dihydroxy-5-(4-(4-(4-(4-methoxy-2H-1,2,3-triazol-2-yl)phenyl)-2-oxopyridin-1(2H)-yl)-2-(methylsulfonyl)butan-2-yl)-1,3,4,2-dioxazaborol-2-uide, can be prepared according to General Procedure IV using (2R)—N-hydroxy-4-{4-[4-(4-methoxy-2H-1,2,3-triazol-2-yl)phenyl]-2-oxopyridin-1(2H)-yl}-2-methyl-2-(methylsulfonyl)butanamide as starting material and sodium hydroxide as the base.

### Example 31

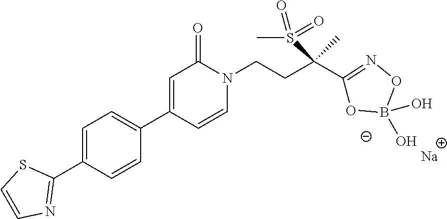

The compound shown above, sodium (R)-2,2-dihydroxy-5-(2-(methylsulfonyl)-4-(2-oxo-4-(4-(thiazol-2-yl)phenyl)pyridin-1(2H)-yl)butan-2-yl)-1,3,4,2-dioxazaborol-2-uide, can be prepared according to General Procedure IV using (2R)—N-hydroxy-2-methyl-2-(methylsulfonyl)-4-{2-oxo-4-[4-(1,3-thiazol-2-yl)phenyl]pyridin-1(2H)-yl}butanamide as starting material and sodium hydroxide as the base.

### Example 32

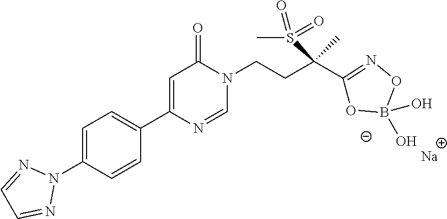

The compound shown above, sodium (R)-5-(4-(4-(4-(2H-1,2,3-triazol-2-yl)phenyl)-6-oxopyrimidin-1(6H)-yl)-2-(methylsulfonyl)butan-2-yl)-2,2-dihydroxy-1,3,4,2-dioxazaborol-2-uide, can be prepared according to General Procedure IV using (R)-4-(4-(4-(2H-1,2,3-triazol-2-yl)phenyl)-6-oxopyrimidin-1(6H)-yl)-N-hydroxy-2-methyl-2-(methylsulfonyl)butanamide as starting material and sodium hydroxide as the base.

### Biological Examples

In order to assess the compounds biological activity, selected in vitro assays were conducted on selected compounds. One of the assays measured the compounds ability to disrupt the synthesis of lipopolysaccharide, LPS, which is a component of the outer membrane of Gram-negative bacteria. Disruption of this synthesis is lethal to the bacteria. The assay determined the compound's ability to inhibit LpxC, which is the first enzyme in the biosynthetic pathway for LPS (measured as IC₅₀). Additionally, MICs (minimal inhibitory concentrations) were determined for several bacteria. The specific protocols are described below:

A) IC₅₀ Assay, LpxC Enzyme from P. aeruginosa (Labeled as PA LpxC Enzyme IC₅₀):

IC₅₀ determination in the LpxC enzyme assay was carried out in a similar manner to that described by Malikzay et al in the 2006 Poster, Screening LpxC (UDP-3-O—(R-3-hydroxymyristoyl)-GcNAc deacetylase) using BioTrove RapidFire HTS Mass Spectrometry (aNew Lead Discovery and blnflammation and Infectious Disease, cStructural Chemistry, Schering-Plough Research Institute, Kenilworth, N.J. 07033, (BioTrove, Inc. 12 Gill St., Suite 4000, Woburn, Mass. 01801). Briefly, Pseudomonas aeruginosa LpxC enzyme (0.1 nM) purified from E. coli-overexpressing bacteria was incubated at 25° C. in a final volume of 50 ul containing 0.5 uM UDP-3-O—(R-3-hydroxydecanoyl)-N-acetylglucosamine, 1 mg/mL BSA, and 50 mM sodium phosphate buffer, pH 8.0 in the presence and absence of inhibitor compound. At the end of 1 hour, 5 ul of 1 N HCl was added to stop the enzyme reaction, the plates were centrifuged, and then processed with the BioTrove Rapidfire HTMS Mass Spectrometry System. A no-enzyme control was used in calculating the IC₅₀ values from the percent conversion values.

B) MIC determinations: The in vitro antibacterial activity of parent compounds of those described in the Examples was evaluated by minimum inhibitory concentration (MIC) testing according to Clinical and Laboratory Standards Institute (CLSI). See: Clinical and Laboratory Standards Institute. Methods for Dilution Antimicrobial Susceptibility Tests for Bacteria that Grow Aerobically; Approved Standard-Eighth Edition. CLSI document M7-A8 [ISBN 1-56238-689-1]. Clinical and Laboratory Standards Institute, 940 West Valley Road, Suite 1400, Wayne, Pa. 19087-1898 USA, 2006; also Clinical and Laboratory Standards Institute. Performance Standards for Antimicrobial Susceptibility Testing; Twentieth Informational Supplement. CLSI document M100-S20 [ISBN1-56238-716-2]. Clinical and Laboratory Standards Institute.

The MIC determination is a standard laboratory method for evaluating the antibacterial activity of a compound. The MIC represents the lowest drug concentration that inhibits visible growth of bacteria following overnight incubation. In order to determine the MIC value, a range of drug concentrations (e.g. 0.06 μg/mL to 64 μg/mL) are incubated with a defined strain of bacteria. Typically, the drug concentration range is broken down into 2-fold increments (e.g. 0.06 μg/mL, 0.12 μg/mL. 0.25 μg/mL, 0.50 μg/mL, 1.0 μg/mL, etc.) and the various drug concentrations are all individually incubated overnight with approximately the same number of bacteria. The MIC is then determined by visually inspecting the drug effect at each concentration, and identifying the lowest drug concentration that has inhibited bacterial growth as compared to the drug free control. Typically, bacteria continue to grow at drug concentrations lower than the MIC and don't grow at concentrations at and above the MIC.

The MIC values described in Table 2 below were derived from assays wherein each test compound was evaluated in duplicate. In cases where the duplicate values varied by 0-2-fold, the lower of the two values was reported below. Generally speaking, if the duplicate values varied by more than 2-fold, the assay was considered non-valid and was repeated until the variation between duplicate runs was <2-fold. In line with the CLSI guidelines referred to above, both control organisms and reference compounds were utilized in each MIC assay to provide proper quality control. MIC values generated with these control organisms and reference compounds were required to fall within a defined range for the assay to be considered valid and be included herein. Those skilled in the art will recognize that MIC values can and do vary from experiment to experiment. Generally speaking, it should be recognized that MIC values often vary +/−2-fold from experiment to experiment. While a single MIC is reported for each compound and each microorganism, the reader should not conclude that each compound was only tested once. Several of the compounds were subjected to multiple tests. The data reported in Table 2 is reflective of the compounds relative activity and different MICs may have been generated on these occasions in line with the guidelines described above.

The following bacterial strains were used in these MIC determinations:

1) Pseudomonas aeruginosa UI-18: Wild-type, labeled as PA-7 in Table 2;

2) Acinetobacter baumannii/haemolyticus: Multidrug-resistant clinical isolate labeled as AB-3167 in Table 2;

3) Escherichia coli EC-1: VOGEL, mouse virulent labeled as EC-1 in Table 2;

4) Klebsiella pneumoniae: Ciprofloxacin-resistant isolate, expresses extended-spectrum beta-lactamases (ESBL), clinical isolate, labeled as KP-3700 in Tables 2.

Table 2, below, shows the results that were obtained for the parent compounds used to prepare the compounds in Examples 1-32. If a particular table entry is left blank, then the data is not available at the current time.

Column 1 corresponds to the parent compound associated with the Example numbers, column 2 provides the IUPAC name, column 3 provides the results from the LpxC enzyme assay described above, and columns 4-7 provide the MIC data as described above.

TABLE 2

| | | PA: IC50 | AB-3167 | EC-1 | KP-3700 | PA-7 |
|---|---|---|---|---|---|---|
| Example | IUPACNAME | (μM) | (μg/mL) | (μg/mL) | (μg/mL) | (μg/mL) |
| Parent | (R)-4-(4-(4-(2H- | 0.0000482 | >64.0 | 0.06 | 0.125 | 0.5 |
| Compound | 1,2,3-Triazol-2- | | | | | |
| of Examples | yl)phenyl)-2- | | | | | |
| 1,6, 11, | oxopyridin-1(2H)-yl)- | | | | | |
| 16, 21-28 | N-hydroxy-2-methyl- | | | | | |
| | 2-(methylsulfonyl) | | | | | |
| | butanamide | | | | | |
| Parent | (2R)-4-[4-(2,3- | 0.000166 | >64.0 | 0.015 | 2 | 1 |
| Compound | difluoro-4- | | | | | |
| of Examples | methoxyphenyl)-2- | | | | | |
| 2, 7, 12, | oxopyridin-1(2H)-yl]- | | | | | |
| 17, 29 | N-hydroxy-2-methyl- | | | | | |
| | 2-(methylsulfonyl) | | | | | |
| | butanamide | | | | | |
| Parent | (2R)-N-hydroxy-4- | 0.000125 | >64.0 | 0.03 | 0.25 | 0.5 |
| Compound | {4-[4-(4-methoxy- | | | | | |
| of Examples | 2H-1,2,3-triazol-2- | | | | | |
| 3, 8, 13, | yl)phenyl]-2- | | | | | |
| 18, 30 | oxopyridin-1(2H)-yl}- | | | | | |
| | 2-methyl-2- | | | | | |
| | (methylsulfonyl) | | | | | |
| | butanamide | | | | | |
| Parent | (2R)-N-hydroxy-2- | 0.000176 | >64.0 | 0.06 | 0.125 | 0.5 |
| Compound | methyl-2- | | | | | |
| of Examples | (methylsulfonyl)-4- | | | | | |
| 4, 9, 14, | {2-oxo-4-[4-(1,3- | | | | | |
| 19, 31 | thiazol-2- | | | | | |
| | yl)phenyl]pyridin- | | | | | |
| | 1(2H)-yl}butanamide | | | | | |
| Parent | (2R)-N-hydroxy-2- | 0.000675 | >64.0 | 0.06 | 0.25 | 0.5 |
| Compound | methyl-2- | | | | | |
| of Examples | (methylsulfonyl)-4- | | | | | |
| 5, 10, 15, | {6-oxo-4-[4-(2H- | | | | | |
| 20, 32 | 1,2,3-triazol-2- | | | | | |
| | yl)phenyl] pyrimidin- | | | | | |
| | 1(6H)-yl}butanamide | | | | | |

We claim:

1. A compound of Formula (1), and stereoisomers thereof; 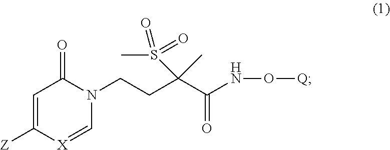
wherein Q is selected from the group consisting of —P(O)(OH)₂, —P(O)(OH)(O⁻M⁺), —P(O)(O⁺M⁺)₂ and —P(O)(O⁻)₂M²⁺;
X is CH or N;
Z is selected from the group consisting of 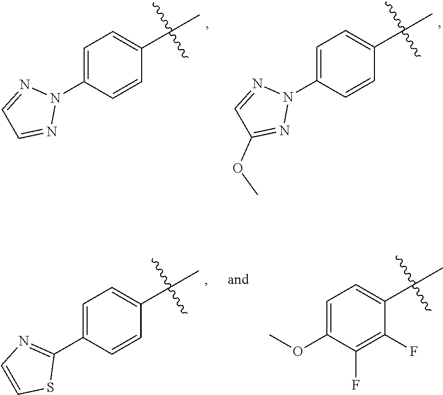 [SMILES: *c1ccc(-n2nccn2)cc1 ; *c1ccc(-n2ncc(OC)n2)cc1 ; *c1ccc(-c2nccs2)cc1 ; *c1ccc(OC)c(F)c1F]
M⁺ at each occurrence is a pharmaceutically acceptable monovalent cation; and
M²⁺ is a pharmaceutically acceptable divalent cation.

2. The compound of Formula (1) of claim 1 that is a compound of Formula (1a)

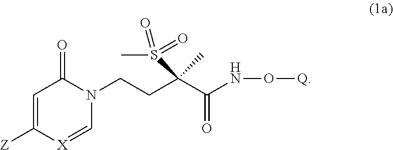

3. The Formula (1a) compound of claim 2 wherein X is CH and Z is

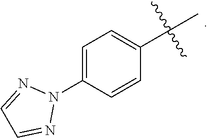 [SMILES: *c1ccc(-n2nccn2)cc1]

4. The Formula (1a) compound of claim 2 wherein Q is —P(O)(OH)₂;
—P(O)(OH)(O⁻M⁺); —P(O)(O⁻M⁺)₂; or —P(O)(O⁻)₂M²⁺; M⁺ at each occurrence is independently selected from the group consisting of Li⁺, K⁺, and Na⁺, or M+ at each occurrence is a pharmaceutically acceptable monovalent cation independently selected from NH₄⁺, NH₃⁺C(CH₂OH)₃, NH₂⁺(CH₂CH₃)₂; NH₂⁺(CH₂CH₃)₂; pyrrolidinium; and glycinium; and wherein M²⁺ is selected from the group consisting of Ca²⁺, Mg²⁺ and Zn²⁺.

5. A compound of Formula (2), and stereoisomers thereof, 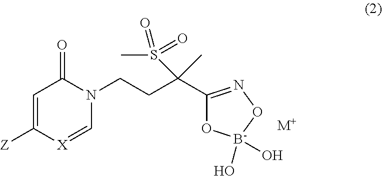
wherein X is CH;
Z is selected from the group consisting of 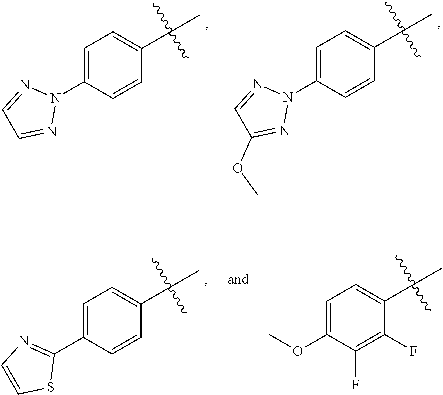 [SMILES: *c1ccc(-n2nccn2)cc1 ; *c1ccc(-n2ncc(OC)n2)cc1 ; *c1ccc(-c2nccs2)cc1 ; *c1ccc(OC)c(F)c1F]
and M⁺ is a pharmaceutically acceptable monovalent cation.

6. A compound of claim 5, that is a compound of Formula (2a) 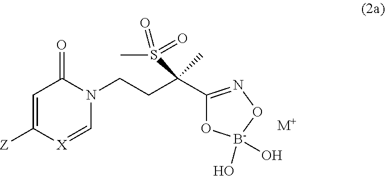
and wherein Z is

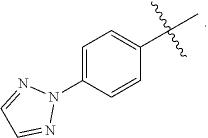 [SMILES: *c1ccc(-n2nccn2)cc1]

7. The compound of claim 6, wherein M⁺ is selected from the group consisting of Li⁺, K⁺, and Na⁺, NH₄⁺, NH₃⁺C(CH₂OH)₃, NH₂⁺(CH₂CH₃)₂, NH₂⁺(CH₂CH₃)₂, pyrrolidinium, and glycinium.

8. A pharmaceutical composition comprising a compound of of claim 1 in admixture with at least one pharmaceutically acceptable excipient, diluent or carrier.

9. A method for treating a bacterial infection in a patient, in need thereof, the method comprising administering a therapeutically effective amount of a compound of claim 1 to a patient, wherein the therapeutically effective amount of the compound is administered orally, topically, or by injection.

10. The method of claim 9 wherein the bacterial infection is a Gram-negative bacterial infection.

11. The method of claim 10 wherein the Gram-negative bacterial infection is caused by a Gram-negative bacteria selected from the group consisting of Mannheimia haemolytica, Pasteurella multocida, Histophilus somni, Actinobacillus pleuropneumoniae, Salmonella enteritidis, Salmonella gallinarium, Lawsonia intracellularis, Brachyspira hyodysenteriae, Brachyspira pilosicoli, Acinetobacter baumannii, Acinetobacter spp., Citrobacter spp., Enterobacter aerogenes, Enterobacter cloacae, Escherichia coli, Klebsiella oxytoca, Klebsiella pneumoniae, Serratia marcescens, Stenotrophomonas maltophilia, and Pseudomonas aeruginosa.

12. The method of claim 10 wherein the Gram-negative bacterial infection is selected from the group consisting of respiratory infection, gastrointestinal infection, nosocomial pneumonia, urinary tract infection, bacteremia, sepsis, skin infection, soft-tissue infection, intraabdominal infection, lung infection, endocarditis, diabetic foot infection, osteomyelitis and central nervous system infection.

13. A method for treating a bacterial infection in a patient, in need thereof, the method comprising administering a therapeutically effective amount of a compound claim 5 to a patient, wherein the therapeutically effective amount of the compound is administered orally, topically, or by injection.

14. The method of claim 13 wherein the bacterial infection is a Gram-negative bacterial infection.

15. A compound selected from the group consisting of
(R)-4-(4-(4-(2H-1,2,3-triazol-2-yl)phenyl)-2-oxopyridin-1(2H)-yl)-2-methyl-2-(methylsulfonyl)butanamido phosphate, disodium salt;
(R)-4-(4-(4-(2H-1,2,3-triazol-2-yl)phenyl)-2-oxopyridin-1(2H)-yl)-2-methyl-2-(methylsulfonyl)butanamido phosphate, diammonium salt;
(R)-4-(4-(4-(2H-1,2,3-triazol-2-yl)phenyl)-2-oxopyridin-1(2H)-yl)-2-methyl-2-(methylsulfonyl)butanamido phosphate, dipotassium salt;
(R)-4-(4-(4-(2H-1,2,3-triazol-2-yl)phenyl)-2-oxopyridin-1(2H)-yl)-2-methyl-2-(methylsulfonyl)butanamido phosphate, dilithium salt;
(R)-4-(4-(4-(2H-1,2,3-triazol-2-yl)phenyl)-2-oxopyridin-1(2H)-yl)-2-methyl-2-(methylsulfonyl)butanamido phosphate, calcium salt;
(R)-4-(4-(4-(2H-1,2,3-triazol-2-yl)phenyl)-2-oxopyridin-1(2H)-yl)-2-methyl-2-(methylsulfonyl)butanamido phosphate, magnesium salt;
(R)-4-(4-(4-(2H-1,2,3-triazol-2-yl)phenyl)-2-oxopyridin-1(2H)-yl)-2-methyl-2-(methylsulfonyl)butanamido phosphate, zinc salt;
(R)-4-(4-(4-(2H-1,2,3-triazol-2-yl)phenyl)-2-oxopyridin-1(2H)-yl)-2-methyl-2-(methylsulfonyl)butanamido phosphate, pyrrolidine salt;
(R)-4-(4-(4-(2H-1,2,3-triazol-2-yl)phenyl)-2-oxopyridin-1(2H)-yl)-2-methyl-2-(methylsulfonyl)butanamido phosphate, tris-(hydroxymethyl)methylamine salt;
(R)-4-(4-(4-(2H-1,2,3-triazol-2-yl)phenyl)-2-oxopyridin-1(2H)-yl)-2-methyl-2-(methylsulfonyl)butanamido phosphate, diethylamine salt;
(R)-4-(4-(4-(2H-1,2,3-triazol-2-yl)phenyl)-2-oxopyridin-1(2H)-yl)-2-methyl-2-(methylsulfonyl)butanamido phosphate, glycine salt; and other pharmaceutically acceptable salts thereof; and a boronate prodrug of (R)-4-(4-(4-(2H-1,2,3-triazol-2-yl)phenyl)-2-oxopyridin-1(2H)-yl)-N-hydroxy-2-methyl-2-(methylsulfonyl) butanamide, and pharmaceutically acceptable salts thereof.

16. The method of claim 14 wherein the Gram-negative bacterial infection is caused by a Gram-negative bacteria selected from the group consisting of Mannheimia haemolytica, Pasteurella multocida, Histophilus somni, Actinobacillus pleuropneumoniae, Salmonella enteritidis, Salmonella gallinarium, Lawsonia intracellularis, Brachyspira hyodysenteriae, Brachyspira pilosicoli, Acinetobacter baumannii, Acinetobacter spp., Citrobacter spp., Enterobacter aerogenes, Enterobacter cloacae, Escherichia coli, Klebsiella oxytoca, Klebsiella pneumoniae, Serratia marcescens, Stenotrophomonas maltophilia, and Pseudomonas aeruginosa.

17. A pharmaceutical composition comprising a compound of claim 5 in admixture with at least one pharmaceutically acceptable excipient, diluent or carrier.

18. A pharmaceutical composition comprising a compound of claim 15 in admixture with at least one pharmaceutically acceptable excipient, diluent or carrier.

19. A method for treating a bacterial infection in a patient, in need thereof, the method comprising administering a therapeutically effective amount of a compound claim 15 to a patient, wherein the therapeutically effective amount of the compound is administered orally, topically, or by injection.

20. The method of claim 19 wherein the bacterial infection is caused by a Gram-negative bacteria selected from the group consisting of Mannheimia haemolytica, Pasteurella multocida, Histophilus somni, Actinobacillus pleuropneumoniae, Salmonella enteritidis, Salmonella gallinarium, Lawsonia intracellularis, Brachyspira hyodysenteriae, Brachyspira pilosicoli, Acinetobacter baumannii, Acinetobacter spp., Citrobacter spp., Enterobacter aerogenes, Enterobacter cloacae, Escherichia coli, Klebsiella oxytoca, Klebsiella pneumoniae, Serratia marcescens, Stenotrophomonas maltophilia, and Pseudomonas aeruginosa.
